(19) United States
(12) Patent Application Publication
(10) Pub. No.: US 2021/0113466 A1
(43) Pub. Date: Apr. 22, 2021

(54) NON-VIRAL, NON-CATIONIC NANOPARTICLES AND USES THEREOF

(71) Applicant: Children's Medical Center Corporation, Boston, MA (US)

(72) Inventors: Marsha A. Moses, Brookline, MA (US); Peng Guo, Boston, MA (US); Jiang Yang, West Lafayette, IN (US); Debra Auguste, Briarcliff Manor, NY (US)

(73) Assignee: Children's Medical Center Corporation, Boston, MA (US)

(21) Appl. No.: 16/494,568

(22) PCT Filed: Mar. 16, 2018
(86) PCT No.: PCT/US18/22890

§ 371 Date: Sep. 16, 2019

Related U.S. Application Data
(60) Provisional application No. 62/472,104, filed on Mar. 16, 2017.

(51) Int. Cl. A61K 9/127; A61K 47/68; A61K 47/69; C12N 15/88; C12N 15/90

(52) U.S. Cl. CPC A61K 9/1272; A61K 47/6845; A61K 47/6913; B82Y 5/00; C12N 15/88; C12N 15/90; A61K 47/6949

(57) ABSTRACT

Some aspects of the present disclosure provide nanoparticles comprising a non-cationic liposome with ligands conjugated to its surface and a hydrogel encapsulated in the liposome. In some embodiments, the nanoparticle is used as a delivery system to deliver an agent (e.g., a therapeutic agent or a genome-editing agents) to a cell (e.g., a diseased cell such as a cancer cell). The ligands on the surface of the cationic liposome targets the liposome to cells that express proteins targeted by the ligands on their surface. Methods of treating diseases and disorders, as well as methods of genome-editing are also provided.

### GOVERNMENT SUPPORT

This invention was made with government support under grants R01CA185530 and 1DP2CA174495 awarded by the National Institutes of Health. The government has certain rights in the invention.

### BACKGROUND

To date, many therapeutic and diagnostic agent delivery systems involve the use of either viral vectors or cationic polymer/lipid based materials. However, the human safety concern of viral vectors and the toxicity of cationic polymer/lipid significantly limit the clinical application potentials of these delivery systems.

### SUMMARY

Provided herein are non-viral, non-cationic nanoparticles for the delivery of agents (e.g., therapeutic agents) to a target cell. The nanoparticles comprises a non-cationic liposome with a hydrogel interior core. The hydrogel core enhances the encapsulation efficiency and ratio of the agents to be delivered. Further, the nanoparticles is able to distinguish the target cell from other cell types due to ligands conjugated to its surface that binds specifically to cell surface proteins on the target cell. In some embodiments, the nanoparticles of the present disclosure are used to deliver gene editing agents (e.g., CRISPR/Cas9 gene editing system) into a target cell (e.g., a cancer cell).

Some aspects of the present disclosure provide nanoparticles containing: (i) a non-cationic liposome; (ii) a ligand conjugated to the liposome surface; and (iii) a hydrogel encapsulated in the liposome.

In some embodiments, the non-cationic liposome comprises a neutral lipid. In some embodiments, the non-cationic liposome does not comprise a cationic lipid. In some embodiments, the neutral lipid is 1,2-dioleoyl-sn-glycero-3-phosphocholine (DOPC). In some embodiments, the nanoparticle comprises an anionic lipid. In some embodiments, the liposome further comprises a pH-responsive lipid. In some embodiments, the pH-responsive lipid comprises 1,2-dioleoyl-3-dimethylammoniumpropane (DODAP).

In some embodiments, the liposome further comprises a functionalized lipid. In some embodiments, the functionalized lipid is a lipid-polymer conjugate. In some embodiments, the lipid-polymer conjugate is a lipid-polyethylene glycol (PEG) conjugate. In some embodiments, the functionalized lipid comprises a carboxylic acid at the distal end of the lipid. In some embodiments, the functionalized lipid is 1,2-distearoyl-sn-glycero-3-phosphoethanolamine-N-[carboxy(polyethylene glycol)-2000]-COOH (DSPE-PEG-COOH).

In some embodiments, the functionalized lipid is up to 10% of total lipids in the liposome. In some embodiments, the liposome comprises DOPC, DODAP, and DSPE-PEG-COOH. In some embodiments, the ratio of DOPC:DODAP:DSPE-PEG-COOH is 85:5:10.

In some embodiments, the hydrogel comprises sodium alginate.

In some embodiments, the nanoparticle has a diameter of no more than 200 nm.

In some embodiments, the ligand targets a cell surface protein. In some embodiments, the ligand is selected from the group consisting of: antibodies, antibody fragments, synthetic peptides, natural ligands, and aptamers.

In some embodiments, the ligand is an antibody. In some embodiments, the antibody is an ICAM-1 antibody. In some embodiments, the nanoparticle further comprises a second ligand conjugated to the liposome surface. In some embodiments, the second ligand targets a second cell surface protein.

In some embodiments, the second ligand is selected from the group consisting of: antibodies, antibodies fragments, synthetic peptides, natural ligands, aptamers. In some embodiments, the second ligand is an antibody. In some embodiments, the second antibody is an EGFR antibody.

In some embodiments, the nanoparticles described herein further contains an agent encapsulated in the liposome. In some embodiments, the agent is a therapeutic agent. In some embodiments, the therapeutic agent is an anti-cancer agent. In some embodiments, the therapeutic agent is selected from the group consisting of: small molecules, oligonucleotides, polypeptides, and combinations thereof.

In some embodiments, the agent comprises a genome-editing agent. In some embodiments, the agent comprises a nucleic acid encoding a Cas9 protein and a guide RNA (gRNA). In some embodiments, the agent comprises an isolated Cas9/gRNA complex.

In some embodiments, the gRNA targets the Cas9 protein to a target gene. In some embodiments, the Cas9 edits the target gene. In some embodiments, the target gene is an oncogene. In some embodiments, the oncogene is lipocalin 2 (Lcn2). In some embodiments, editing of the oncogene by Cas9 inactivates the oncogene.

Compositions comprising the nanoparticles described herein are provided.

Other aspects of the present disclosure provide delivery systems, containing: (i) a non-cationic liposome; (ii) a ligand conjugated to the liposome surface; (iii) a hydrogel encapsulated in the liposome; and (iv) a genome-editing agent encapsulated in the liposome.

In some embodiments, the non-cationic liposome comprises a neutral lipid. In some embodiments, the non-cationic liposome does not comprise a cationic lipid. In some embodiments, the neutral lipid is 1,2-dioleoyl-sn-glycero-3-phosphocholine (DOPC). In some embodiments, the nanoparticle comprises an anionic lipid. In some embodiments, the liposome further comprises a pH-responsive lipid. In some embodiments, the pH-responsive lipid comprises 1,2-dioleoyl-3-dimethylammoniumpropane (DODAP). In some embodiments, the liposome further comprises a functionalized lipid. In some embodiments, the functionalized lipid is a lipid-polymer conjugate. In some embodiments, the lipid-polymer conjugate is a lipid-polyethylene glycol (PEG) conjugate. In some embodiments, the functionalized lipid comprises a carboxylic acid at the distal end of the lipid. In some embodiments, the functionalized lipid is 1,2-distearoyl-sn-glycero-3-phosphoethanolamine-N-[carboxy(polyethylene glycol)-2000]-COOH (DSPE-PEG-COOH).

In some embodiments, the functionalized lipid is up to 10% of total lipids in the liposome. In some embodiments, the liposome comprises DOPC, DODAP, and DSPE-PEG-COOH. In some embodiments, the ratio of DOPC:DODAP:DSPE-PEG-COOH is 85:5:10. In some embodiments, the hydrogel comprises sodium alginate.

In some embodiments, the nanoparticle has a diameter of less than 200 nm.

In some embodiments, the ligand targets a cell surface protein. In some embodiments, the ligand is selected from the group consisting of: antibodies, antibodies fragments, synthetic peptides, natural ligands, aptamers. In some embodiments, the ligand is an antibody. In some embodiments, the antibody is an ICAM-1 antibody. In some embodiments, the nanoparticle further comprises a second ligand conjugated to the liposome surface. In some embodiments, the second ligand targets a second cell surface protein. In some embodiments, the second ligand is selected from the group consisting of: antibodies, antibodies fragments, synthetic peptides, natural ligands, aptamers. In some embodiments, the second ligand is an antibody. In some embodiments, the second antibody is an EGFR antibody.

In some embodiments, the genome-editing agent comprises a nucleic acid encoding a Cas9 protein and a guide RNA (gRNA). In some embodiments, the genome-editing agent comprises an isolated Cas9/gRNA complex. In some embodiments, the gRNA targets the Cas9 protein to a target gene. In some embodiments, the Cas9 edits the target gene.

Compositions comprising the delivery systems described herein are also provided.

Other aspects of the present disclosure provide methods of delivering an agent to a cell, including contacting the cell with the nanoparticle or the delivery system described herein, wherein the cell expresses a surface protein targeted by the ligand on the nanoparticle, and wherein the contacting results in delivery of the agent to the cell.

In some embodiments, the cell is a mammalian cell. In some embodiments, the cell is a human cell. In some embodiments, the cell is a cultured cell. In some embodiments, the cell is a cell in vivo in a subject. In some embodiments, the cell is a cancer cell. In some embodiments, the cancer cell is a triple negative breast cancer cell (TNBC).

Further provided herein are methods of treating a disease or disorder, the method including administering a therapeutically effective amount of a delivery system to a subject in need thereof, wherein the delivery system comprises the nanoparticle nanoparticles described herein and a therapeutic agent encapsulated in the nanoparticle.

In some embodiments, the disease or disorder is cancer. In some embodiments, the cancer is selected from the group consisting of: breast cancer, pancreatic cancer, brain and central nervous system cancer, skin cancer, ovarian cancer, leukemia, endometrial cancers, bone, cartilage and soft tissue sarcomas, lymphoma, neuroblastoma, nephroblastoma, retinoblastoma, and gonadal germ cell tumors. In some embodiments, the cancer is triple negative breast cancer (TNBC). In some embodiments, the delivery system is administered orally, parenterally, intramuscularly, intranasally, intratracheal, intracerebroventricularly, intravenously, or intraperitoneally.

Yet other aspects of the present disclosure provide methods of editing a target gene in the genome of a subject, the method including administering to the subject an effective amount of the delivery system described herein. In some embodiments, the target gene is associated with a disease or disorder, and wherein editing the target gene results in an edited gene that is not associated with the disease or disorder.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIGS. 1A-1H show the design of targeting nanolipogel (TNLG) (FIG. 1A), the size distribution (FIG. 1B), TEM images of nanoliposome (without hydrogel) and nanolipogel (with hydrogel) (FIGS. 1C and 1D, respectively, the scales bars are 1 μm and 100 nm (inset)). The encapsulation efficiency of CRISPR-Cas9 plasmid (FIG. 1E), siRNA (FIG. 1F), Herceptin (FIG. 1G), and Rhodamine-dextran (FIG. 1H) in TNLGs is also shown.

FIGS. 2A-2B show the serum stability (FIG. 2A) and cytotoxicity (FIG. 2B) of TNLGs.

FIGS. 3A-3C show the gene editing efficiency of Lcn2 CRISPR-Cas9 knockout plasmid encapsulating TNLGs in MDA-MB-231 (FIG. 3A), MDAMB-157 (FIG. 3B), and MDA-MB-436 (FIG. 3C) cells.

FIGS. 4A-4E show the therapeutic effects of TNLGs with Lcn2 CRISPR-Cas9 knockout plasmid. FIG. 4A shows MDA-MB-231 cell proliferation treated with TNLGs or control groups. Representative images (FIG. 4B) and quantified cell numbers (FIG. 4C) of MDA-MB-231 cell transwell migration treated TNLGs or control groups. Cell migration tracks (FIG. 4D) and quantified cell speed (FIG. 4E) of MDA-MB-231 cells treated with TNLGs or control groups are also shown.

FIG. 5. Orthotopic MDA-MB-231 tumor accumulation of ICAM1-targeted, near-infrared dye DiR-labelled nanolipogels (ICAM1-DiR-Lipogel) in comparison with non-specific IgG-DiR-Lipogel. (n=3 per group).

FIG. 6. Systematic toxicity of ICAM1-targeted nanolipogel (ICAM1-Lipogel) in nude mice in comparison with PBS (sham group) at post-48 h intravenous injection. Serum levels of AST, ALT, Creatinine, and BUN (n=4-5 per group). NS, not significant.

### DETAILED DESCRIPTION OF CERTAIN EMBODIMENTS

Provided herein are novel non-viral, non-cationic nanoparticles, their use in delivering agents (e.g., therapeutic agents) into a target cell (e.g., cancer cell), and methods of making them. The nanoparticles comprises a non-cationic liposome with a hydrogel interior core. The hydrogel core enhances the encapsulation efficiency and ratio of the agents to be delivered. Further, the nanoparticles is able to distinguish the target cell from other cell types due to ligands conjugated to its surface that binds specifically to cell surface proteins on the target cell. In some embodiments, the nanoparticles of the present disclosure are used to deliver gene editing agents (e.g., CRISPR/Cas9 gene editing system) into a target cell (e.g., a cancer cell).

Some aspects of the present disclosures relate to non-viral, non-cationic nanoparticles. A “nanoparticle” generally refers to a particle having a diameter from about 10 nm up to, but not including, about 1 micron. In some embodiments, the nanoparticle is from 100 nm to, but not including, about 1 micron. The nanoparticles of the present disclosure generally have a spherical shape. A “non-viral” nanoparticle means the nanoparticle does not rely one viral proteins (e.g., viral capsid proteins) for its assembly.

The nanoparticles of the present disclosure comprise a non-cationic liposome, a ligand conjugated to the liposome surface, and a hydrogel encapsulated in the liposome. A “liposome” is a microscopic vesicle having at least one concentric lipid bilayers. In some embodiments, a liposome has one lipid bilayer. Structurally, liposomes range in size and shape from long tubes to spheres, with dimensions from a few hundred Angstroms to fractions of a millimeter. In some embodiments, the liposome is a sphere. Typically, liposomes can be divided into three categories based on their overall size and the nature of the lamellar structure. The three classifications, as developed by the New York Academy Sciences Meeting (Liposomes and Their Use in Biology and Medicine, December 1977, incorporated herein by reference), are multi-lamellar vesicles (MLVs), small uni-lamellar vesicles (SUVs) and large uni-lamellar vesicles (LUVs). SUVs range in diameter from approximately 20 to 100 nm and consist of a single lipid bilayer surrounding an aqueous compartment. Large unilamellar vesicles can also be prepared in sizes from about 100 nm to a few micrometers (e.g., 30 μm) in diameter. While unilamellar vesicles are single compartmental vesicles of fairly uniform size, MLVs vary greatly in size up to 10,000 nm, or thereabouts, are multi-compartmental in their structure and contain more than one bilayer. The liposomes of the present disclosure are unilamellar vesicles. Unilamella Liposomes comprise a completely closed lipid bilayer with an encapsulated aqueous volume.

Liposomes have typically been prepared using the process of Bangham et al., (1965 J. Mol. Biol., 13: 238-252), whereby lipids suspended in organic solvent are evaporated under reduced pressure to a dry film in a reaction vessel. An appropriate amount of aqueous phase is then added to the vessel and the mixture agitated. The mixture is then allowed to stand, essentially undisturbed for a time sufficient for the multilamellar vesicles to form. The aqueous phase entrapped within the liposomes may contain bioactive agents, for example drugs, hormones, proteins, dyes, vitamins, or imaging agents, among others.

Liposomes may be reproducibly prepared using a number of currently available techniques. The types of liposomes which may be produced using a number of these techniques include small unilamellar vesicles (SUVs) (e.g., as described in Papahadjapoulous and Miller, Biochem. Biophys. Acta., 135, p. 624-638 (1967), incorporated herein by reference), reverse-phase evaporation vesicles (REV) (e.g., U.S. Pat. No. 4,235,871 issued Nov. 25, 1980, incorporated herein by reference), stable plurilamellar vesicles (SPLV) (e.g., U.S. Pat. No. 4,522,803, issued Jun. 11, 1985, incorporated herein by reference), and large unilamellar vesicles produced by an extrusion technique (e.g., as described in U.S. patent application Ser. No. 622,690, filed Jun. 20, 1984, Cullis et. al., entitled “Extrusion Technique for Producing Unilamellar Vesicles”, incorporated herein by reference).

The lipid bilayer of the liposome is composed of two layers of lipid molecules organized in two sheets. Biological bilayers are usually composed of amphiphilic phospholipids that have a hydrophilic phosphate head and a hydrophobic tail consisting of two fatty acid chains. Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. The two components are joined together by a glycerol. molecule. The phosphate groups can be modified with simple organic molecules such as choline.

When phospholipids are exposed to water, they self-assemble into a two-layered sheet with the hydrophobic tails pointing toward the center of the sheet, resulting in two “leaflets” that are each a single molecular layer. The center of this bilayer contains almost no water and excludes molecules like sugars or salts that dissolve in water. The assembly process is driven by interactions between hydrophobic molecules (also called the hydrophobic effect). An increase in interactions between hydrophobic molecules (causing clustering of hydrophobic regions) allows water molecules to bond more freely with each other, increasing the entropy of the system. This complex process includes non-covalent interactions such as van der Waals forces, electrostatic and hydrogen bonds. Phospholipids with certain head groups can alter the surface chemistry of a bilayer and can, for example, serve as signals as well as “anchors” for other molecules in the membranes of cells.

The lipid bilayer of liposomes typical contain vesicle-forming lipids. The specified degree of fluidity or rigidity of the final liposome complex depends on the lipid composition of the outer layer. In some embodiments, lipids in the lipid bilayers of liposomes are neutral (cholesterol) or bipolar and include phospholipids, such as phosphatidylcholine (PC), phosphatidylethanolamine (PE), phosphatidylinositol (PI), and sphingomyelin (SM) and other type of bipolar lipids including but not limited to dioleoylphosphatidylethanolamine (DOPE), with a hydrocarbon chain length in the range of 14-22, and saturated or with one or more double C═C bonds. Examples of lipids capable of producing a stable liposome, alone, or in combination with other lipid components include, without limitation phospholipids, such as hydrogenated soy phosphatidylcholine (HSPC), lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, di stearoylphosphatidylethanolamine (DSPE), dioleoylphosphatidylcholine (DOPC), dipalmitoylphosphatidylcholine (DPPC), palmitoyloleoylphosphatidylcholine (POPC), palmitoyloleoylphosphatidylethanolamine (POPE) and dioleoylphosphatidylethanolamine 4-(N-maleimido-methyl)cyclohexane-1-carboxylate (DOPE-mal). Additional non-phosphorous containing lipids that can become incorporated into liposomes include stearylamine, dodecylamine, hexadecylamine, isopropyl myristate, triethanolamine-lauryl sulfate, alkyl-aryl sulfate, acetyl palmitate, glycerol ricinoleate, hexadecyl stereate, amphoteric acrylic polymers, polyethyloxylated fatty acid amides, and the cationic lipids mentioned above (DDAB, DODAC, DMRIE, DMTAP, DOGS, DOTAP (DOTMA), DOSPA, DPTAP, DSTAP, DC-Chol). Negatively charged lipids include phosphatidic acid (PA), dipalmitoylphosphatidylglycerol (DPPG), dioleoylphosphatidylglycerol and (DOPG), dicetylphosphate that are able to form vesicles.

The liposome of the present disclosure is a non-cationic liposome. A “non-cationic liposome” is a liposome that does not have an overall positive charge. For example, a non-cationic liposome may have an overall neutral charge (i.e., no charge) or an overall negative charge. In some embodiments, a non-cationic liposome may contain neutral lipids, anionic lipids and/or cationic lipids, so long as the overall charge of the liposome remains neutral or negative. In some embodiments, a non-cationic liposome contains cationic lipids. In some embodiments, a non-cationic liposome does not contain cationic lipids.

A “neutral lipid” is a lipid molecule (e.g., a phospholipid molecule) lacking charged groups or having an overall neutral charge. Neutral lipids that may be used in accordance with the present disclosure include, without limitation: dioleoylphosphatidylcholine, dioleoylphosphatidylethanolamine, dilinoleoylphosphatidylcholine, di stearoylphophatidylethanolamine, di stearoylphosphatidylcholine, dipalmitoylphosphatidylcholine, dipalmitoyl phosphatidylethanolamine, egg phosphatidylcholine, dilauryloylphosphatidylcholine, dimyristoylphosphatidylcholine, 1-myristoyl-2-palmitoyl phosphatidylcholine, 1-palmitoyl-2-myristoyl phosphatidylcholine, 1-palmitoyl-2-stearoyl phosphatidylcholine, 1-stearoyl-2-palmitoyl phosphatidylcholine, dimyristyl phosphatidylcholine, 1,2-distearoyl-sn-glycero-3-phosphocholine, 1,2-diarachidoyl-sn-glycero-3-phosphocholine, 1,2-dieicosenoyl-sn-glycero-3-phosphocholine, palmitoyloeoyl phosphatidylcholine, dimyristoyl phosphatidylethanolamine, palmitoyloeoyl phosphatidylethanolamine, cholesterol, 14Z,17Z,20Z,23Z,26Z,29Z-dotriacontahexaenoic acid, N-oleoylglycine, N-arachidonoylglycine, N-palmitoylglycine, 2-hydroxyoleic acid (sodium salt), 5-(palmitoyloxy)octadecanoic acid, 9-(palmitoyloxy)octadecanoic acid, 9-[(13,13,14,14,15,15,16,16,16-d9)palmitoyl)hydroxy]-stearic acid, 5-[(13,13,14,14,15,15,16,16,16-d9)palmitoyl)hydroxy]-stearic acid, Polyprenal, Dolichol, Coenzyme Q8 (E. coli), Coenzyme Q6, Prostaglandin B1, Prostaglandin A1, Prostaglandin F1β, Prostaglandin F1α, Prostaglandin E1, 1,2-diacyl-3-O-(α-D-glucopyranosyl)-sn-glycerol (E. coli), Monogalactosyldiacylglycerol (Plant), Digalactosyldiacylglycerol (Plant), sulfoquinovosyldiacylglycerol, 1-O-hexadecyl-sn-glycerol (HG), 1-O-hexadecyl-2-O-methyl-sn-glycerol (PMG), 1-O-hexadecyl-2-acetyl-sn-glycerol (HAG), Monogalactosyldiacylglycerol (Plant), Digalactosyldiacylglycerol (Plant), sulfoquinovosyldiacylglycerol, 1,2-dipalmitoyl-sn-glycero-3-O-4′-(N,N,N-trimethyl)-homoserine, 1,2-dipalmitoyl-sn-glycero-3-O-4′-[N,N,N-trimethyl(d9)]-homoserine, campest-5-en-3β-ol, campesterol-d6, β-sitostanol, 22,23-dihydrostigmasterol, (24-ethyl)-heptadeuteriostigmast-5-en-3β-ol, stigmasta-5,22-dien-3-ol, 1,2-dipalmitoyl ethylene glycol, 1-2-dioleoyl ethylene glycol, 1-O-hexadecyl-sn-glycerol (HG), 1,2-dioctanoyl-sn-glycerol, 1,2-didecanoyl-sn-glycerol, 1,2-dilauroyl-sn-glycerol, 1,2-dimyristoyl-sn-glycerol, 1,2-dipalmitoyl-sn-glycerol, 1,2-di-O-phytanyl-sn-glycerol, 1-2-dioleoyl-sn-glycerol, 1-palmitoyl-2-oleoyl-sn-glycerol, and 1-stearoyl-2-linoleoyl-sn-glycerol. In some embodiments, the neutral lipid is 1,2-dioleoyl-sn-glycero-3-phosphocholine (DOPC).

An “anionic lipid” is a lipid molecule (e.g., a phospholipid molecule) with an overall negative charge. In some embodiments, an anionic lipid is a phospholipid with a negatively charged head group. Anionic lipids that may be used in accordance with the present disclosure include, without limitation: L-α-phosphatidylglycerol, L-α-phosphatidylserine, L-α-lysophosphatidylserine, L-alpha-lysophosphatidylinositol, L-α-phosphatidylinositol, cyclic phosphatidic acid, and phosphatidic acid.

A “cationic lipid” is a lipid molecule (e.g., a phospholipid molecule) with an overall positive charge. In some embodiments, the cationic lipid is a phospholipid has a positively charged headgroup. In some embodiments, the cationic lipid may be N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP). 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethyl amino-ethane)carbamoyl] cholesterol (DC-Choi); 2,3-dioleoyloxy-N-(2-(sperrninecarboxamido)-ethyl)-N,N-dimethyl-1-propanam-inium trifluoro-acetate (DOSPA), .beta.-alanyl cholesterol, cetyl trimethyl ammonium bromide (CTAB), diC. sub.14-ami dine, N-ferf-butyl-N′-tetradecy 1-3-tetradecylamino-propionami dine, N-(alpha-trimethylammonioacetyl)didodecyl-D-glutamate chloride (TMAG), ditetradecanoyl-N-(trimethylarnmonio-acetyl)diethanolamine chloride, 1,3-diol eoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In some embodiments, the cationic lipids may be 1-[2-(acyloxy)ethyl]2-alkyl(alkenyl)-3-(2-hydroxyethyl)-imidazolinium chloride derivatives, for example, without limitation, 1-[2-(9(Z)-octadecenoyloxy)ethyl]-2-(8(Z)-heptadecenyl-3-(2-hydroxy ethyl)-imidazolinium chloride (DOTIM), and 1-[2-(hexadecanoyloxy)ethyl]-2-pentadecyl-3-(2-hydroxyethyl)imidazolinium chloride (DPTIM). In some embodiments, the cationic lipids may be 2,3-dialkyloxypropyl quaternary ammonium compound derivatives containing a hydroxyalkyl moiety on the quaternary amine, for example, without limitation, 1,2-dioleoyl-3-dimethyl-hydroxy ethyl ammonium bromide (DORI), 1,2-dioleyloxypropyl-3-dimethyl-hydroxy ethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxy propyl-3-dimethyl-hydroxylethy 1 ammonium bromide (DMRIE), 1,2-dipalmityloxypropyl-3-dimethyl-hydroxyethyl ammonium bromide (DPRIE), and 1,2-disteryloxypropyl-3-dimethyl-hydroxy ethyl ammonium bromide (DSRIE). In some embodiments, the cationic lipid may be, without limitation: N1-[2-((1S)-1-[(3-aminopropyl)amino]-4-[di(3-amino-propyl)amino]butylcarboxamido)ethyl]-3,4-di[oleyloxy]-benzamide, 1,2-di-O-octadecenyl-3-trimethylammonium propane (chloride salt), 1,2-dimyristoleoyl-sn-glycero-3-ethylphosphocholine (Tf salt), 1-palmitoyl-2-oleoyl-sn-glycero-3-ethylphosphocholine (chloride salt), 1,2-dioleoyl-sn-glycero-3-ethylphosphocholine (chloride salt), 1,2-distearoyl-sn-glycero-3-ethylphosphocholine (chloride salt), 1,2-dipalmitoyl-sn-glycero-3-ethylphosphocholine (chloride salt), 1,2-dimyristoyl-sn-glycero-3-ethylphosphocholine (chloride salt), 1,2-dilauroyl-sn-glycero-3-ethylphosphocholine (chloride salt), Dimethyldioctadecylammonium (Bromide Salt), 3β-[N—(N′,N′-dimethylaminoethane)-carbamoyl]cholesterol hydrochloride, 1,2-dioleoyl-3-dimethylammonium-propane (DODAP), 1,2-dimyristoyl-3-dimethylammonium-propane, 1,2-dipalmitoyl-3-dimethylammonium-propane, 1,2-distearoyl-3-dimethylammonium-propane, N-(4-carboxybenzyl)-N,N-dimethyl-2,3-bis(oleoyloxy)propan-1-aminium, 1,2-dioleoyl-3-trimethylammonium-propane (methyl sulfate salt), 1,2-dioleoyl-3-trimethylammonium-propane (chloride salt), 1,2-stearoyl-3-trimethylammonium-propane (chloride salt), 1,2-dipalmitoyl-3-trimethylammonium-propane (chloride salt), 1,2-dimyristoyl-3-trimethylammonium-propane (chloride salt), or 1-oleoyl-2-[6-[(7-nitro-2-1,3-benzoxadiazol-4-yl)amino]hexanoyl]-3-trimethylammonium propane (chloride salt).

In some embodiments, the non-cationic liposomes of the present disclosure comprises a pH-responsive lipid. A “pH-responsive lipid” refers to a lipid (e.g., a phospholipid) that contains a moiety that is responsive to pH such that the lipid is neutral at physiological pH (e.g., at a pH of about 7.4) but becomes positively charged when it is in an environment with a pH lower than physiological pH (e.g., at a pH of between 1-7). For example, a lipid having an imidazole moiety, which has a pK of about 6.0, will become predominantly positively charged at pH values less than 6.0. Therefore, in an endosome where the pH is between about 5.0 to about 6.0, the lipid protonates, facilitating uptake and release of the encapsulated cargo into the cytoplasm of the cell (e.g., as described in Xu et al., Biochemistry, 35:5616-5623 (1996)).

Non-limiting, exemplary pH-responsive lipids (e.g., phospholipids) that may be used in accordance with the present disclosure include N-palmitoyl homocysteine, 1,2-dioleoyl-sn-glycero-3-succinate, N-(4-carboxybenzyl)-N,N-dimethyl-2,3-bis(oleoyloxy)propan-1-aminium, 1,2-dioleoyl-3-dimethylammonium-propane (DODAP), 1,2-dimyristoyl-3-dimethylammonium-propane, 1,2-dipalmitoyl-3-dimethylammonium-propane, 1,2-distearoyl-3-dimethylammonium-propane, and N-(4-carboxybenzyl)-N,N-dimethyl-2,3-bis(oleoyloxy)propan-1-aminium. In some embodiments, the liposomes described herein comprises a pH-responsive lipid DODAP.

Liposomes containing pH-responsive lipids (e.g., pH-responsive phospholipids) may be referred to as pH-responsive liposomes. PH-responsive liposomes, when administered to a subject, such as a mammal, for example, a human, are uncharged, which allows for a longer blood circulation time than achieved with charged liposomes. Liposomes that are endocytosed or that reach a specific in vivo region where the pH is lower, become charged as the lipid becomes positively charged. This is due to the liposomes having a pH responsive moiety. This can occur, for example, in a tumor region or in a lysosome.

In some embodiments, the non-cationic liposomes of the present disclosure comprises a functionalized lipid. A “functionalized lipid” is a lipid (e.g., a phospholipid) that contains a reactive (i.e., functionalized) group (e.g., chemical group) that may be used to attach (e.g., covalently or non-covalently) a molecule (e.g., a chemical compound or a biological molecular such as a nucleic acid or a polypeptide) to the lipid. Functionalized lipids and methods of producing them are known in the art, e.g., as described in U.S. Pat. No. 5,556,948, incorporated herein by reference. In some embodiments, the functionalized lipid is a lipid-polymer conjugate.

A “lipid-polymer conjugate” refers to a lipid linked to a polymer covalently or non-covalently. A “polymer” is a substance that has a molecular structure consisting mainly or entirely of a large number of similar units bonded together, e.g., many synthetic organic materials used as plastics and resins. The polymer may be homopolymers or copolymers. Homopolymers are polymers which have one monomer in their composition. Copolymers are polymers which have more than one type of monomer in their composition. Copolymers may be block copolymers or random copolymers. Block copolymers contain alternating blocks (segments) of different homopolymers. Random copolymers contain random sequences of two or more monomers. A polymer is “soluble” in water if the polymer (either a homopolymer or copolymer) is soluble to at least 5% by weight at room temperature at a polymer size between about 20-150 subunits. A polymer is “soluble” in a polar organic solvent, which may be chloroform, acetonitrile, dimethylformamide, and/or methylene chloride, if the polymer (either a homopolymer or copolymer) is soluble to at least 0.5% by weight at room temperature, at a polymer size between about 20-150 subunits. Types of polymers that may be used to form lipid-polymer conjugates are known in the art, e.g., as described in U.S. Pat. Nos. 5,395,619 and 5,013,556, incorporated herein by reference.

Non-limiting examples of water soluble polymers include polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl-pyrrolidone)polyethylene glycol, propropylene glycol homopolymers, polypropylene oxide/ethylene oxide copolymers, and polyoxyethylated polyols.

Further examples of polymer conjugation include but are not limited to polymers such as polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and α,β-Poly[(2-hydroxyethyl)-DL-aspartamide, and the like, or mixtures thereof. Conjugation to a polymer can improve serum half-life, among other effects. Methods of conjugation are well known in the art, for example, P. E. Thorpe, et al, 1978, Nature 271, 752-755; Harokopakis E., et al., 1995, Journal of Immunological Methods, 185:31-42; S. F. Atkinson, et al., 2001, J. Biol. Chem., 276:27930-27935; and U.S. Pat. Nos. 5,601,825, 5,180,816, 6,423,685, 6,706,252, 6,884,780, and 7,022,673, incorporated herein by reference.

In some embodiments, the lipid-polymer conjugate described herein comprises a lipid (e.g., phospholipid) linked to a polyethylene glyco (PEG). In some embodiments, the lipid is covalently attached to the polymer (e.g., PEG). The polymer may be of any molecular weight, and may be branched or unbranched. In some embodiments, the PEG used in accordance with the present disclosure is linear, unbranched PEG having a molecular weight of from about 1 kilodaltons (kDa) to about 60 kDa (the term “about” indicating that in preparations of PEG, some molecules will weigh more, and some less, than the stated molecular weight). For example, the PEG may have a molecular weight of 1-60, 1-50, 1-40, 1-30, 1-20, 1-10, 1-5, 5-60, 5-50, 5-40, 5-30, 5-20, 5-10, 10-60, 10-50, 10-40, 10-30, 10-20, 20-60, 20-50, 20-40, 20-30, 30-60, 30-50, 30-40, 40-60, 40-50, or 50-60 kDa. In some embodiments, the PEG has a molecular weight of 1, 2, 3, 4, 5, 10, 15, 20, 25, 30, 35, 40, 45, 50, 55, or 60 kDa.

In some embodiments, the functionalized lipid comprises reactive group or functional group at the distal end of the lipid. In some embodiments, the polymer (e.g., PEG) conjugated to the lipid contains a reactive group of function group at the distal end of the lipid. The “distal end” has the common meaning in the art and refers to the end that is away from the lipid bilayer. The reactive group or functional group is on the surface of the liposome, i.e., exposed and accessible to other molecules.

A “reactive group” or “functional group” refers to specific groups (moieties) of atoms or bonds within molecules that are responsible for the characteristic chemical reactions of those molecules. These terms are used interchangeably herein. One example of such reactive group is a “click chemistry handle.” Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “Click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgon cycloaddition; thiol-yne addition; imine formation; and Michael additions (e.g., maleimide addition). Non-limiting examples of a click chemistry handle include an azide handle, an alkyne handle, or an aziridine handle. Azide is the anion with the formula N3-. It is the conjugate base of hydrazoic acid (HN3). N3-is a linear anion that is isoelectronic with CO₂, NCO—, N₂O, NO₂+ and NCF. Azide can be described by several resonance structures, an important one being —N═N+=N—. An alkyne is an unsaturated hydrocarbon containing at least one carbon-carbon triple bond. The simplest acyclic alkynes with only one triple bond and no other functional groups form a homologous series with the general chemical formula CnH₂n-2. Alkynes are traditionally known as acetylenes, although the name acetylene also refers specifically to C₂H₂, known formally as ethyne using IUPAC nomenclature. Like other hydrocarbons, alkynes are generally hydrophobic but tend to be more reactive. Aziridines are organic compounds containing the aziridine functional group, a three-membered heterocycle with one amine group (—NH—) and two methylene bridges (—CH₂—). The parent compound is aziridine (or ethylene imine), with molecular formula C₂H₅N.

Other non-limiting, exemplary reactive groups include: acetals, ketals, hemiacetals, and hemiketals, carboxylic acids, strong non-oxidizing acids, strong oxidizing acids, weak acids, acrylates and acrylic acids, acyl halides, sulfonyl halides, chloroformates, alcohols and polyols, aldehydes, alkynes with or without acetylenic hydrogen amides and imides, amines, aromatic, amines, phosphines, pyridines, anhydrides, aryl halides, azo, diazo, azido, hydrazine, and azide compounds, strong bases, weak bases, carbamates, carbonate salts, chlorosilanes, conjugated dienes, cyanides, inorganic, diazonium salts, epoxides, esters, sulfate esters, phosphate esters, thiophosphate esters borate esters, ethers, soluble fluoride salts, fluorinated organic compounds, halogenated organic compounds, halogenating agents, aliphatic saturated hydrocarbons, aliphatic unsaturated hydrocarbons, hydrocarbons, aromatic, insufficient information for classification, isocyanates and isothiocyanates, ketones, metal hydrides, metal alkyls, metal aryls, and silanes, alkali metals, nitrate and nitrite compounds, inorganic, nitrides, phosphides, carbides, and silicides, nitriles, nitro, nitroso, nitrate, nitrite compounds, organic, non-redox-active inorganic compounds, organometallics, oximes, peroxides, organic, phenolic salts, phenols and cresols, polymerizable compounds, quaternary ammonium and phosphonium salts, strong reducing agents, weak reducing agents, acidic salts, basic salts, siloxanes, inorganic sulfides, organic sulfides, sulfite and thiosulfate salts, sulfonates, phosphonates, organic thiophosphonates, thiocarbamate esters and salts, and dithiocarbamate esters and salts. In some embodiments, the reactive group is a carboxylic acid group.

Non-limiting, exemplary functionalized lipids (e.g., phospholipids) include: 1,2-distearoyl-sn-glycero-3-phosphoethanolamine-N-[amino(polyethylene glycol)], D-lactosyl-β-1,1′N-(6″-azidohexanoyl)-D-erythro-sphingosine, N-(6-azidohexanoyl)-D-erythro-sphingosine, D-galactosyl-β-1,1′N-(6″-azidohexanoyl)-D-erythro-sphingosine, D-gluctosyl-β-1,1′N-(6″-azidohexanoyl)-D-erythro-sphingosine, (2S,3R,E)-2-amino-13-(3-(pent-4-yn-1-yl)-3H-diazirin-3-yl)dodec-4-ene-1,3-diol, Hex-5′-ynyl 3β-hydroxy-6-diazirinyl-5α-cholan-24-oate, 27-norcholest-5-en-25-yn-3β-ol, 27-alkyne cholesterol, 5Z,8Z,11Z,14Z-eicosatetraen-19-ynoic acid, 1,2-distearoyl-sn-glycero-3-phosphoethanolamine-N-[dibenzocyclooctyl(polyethylene glycol)], 1,2-dipalmitoyl-sn-glycero-3-phosphoethanolamine-N-(5-hexynoyl), 1,2-dipalmitoyl-sn-glycero-3-phosphoethanolamine-N-(6-azidohexanoyl), 1,2-dipalmitoyl-sn-glycero-3-phosphoethanolamine-N-dibenzocyclooctyl, 1,2-dioleoyl-sn-glycero-3-phosphoethanolamine-N-dibenzocyclooctyl, 15-hexadecynoic acid, (Z)-octadec-9-en-17-ynoic acid, 9-(3-pent-4-ynyl-3-H-diazirin-3-yl)-nonanoic acid, N-(9-(3-pent-4-ynyl-3-H-diazirin-3-yl)-nonanoyl)-D-erythro-sphingosine, D-galactosyl-β-1,1′N-(9-(3-pent-4-ynyl-3-H-diazirin-3-yl)-nonanoyl)-D-erythro-sphingosine, D-glucosyl-β-1,1′N-(9-(3-pent-4-ynyl-3-H-diazirin-3-yl)-nonanoyl)-D-erythro-sphingosine, 1-palmitoyl-2-(9-(3-pent-4-ynyl-3-H-diazirin-3-yl)-nonanoyl)-sn-glycero-3-phosphocholine, 1-(9-(3-pent-4-ynyl-3-H-diazirin-3-yl)-nonanoyl)-2-oleoyl-sn-glycero-3-phosphocholine, 1,2-dioleyl-sn-glycero-3-phosphoethanolamine-N-(dabsyl), 1,2-dipalmitoyl-sn-glycero-3-phospho((ethyl-1′,2′,3′-triazole)triethyleneglycolmannose), 1,2-Dipalmitoyl-sn-Glycero-3-Phosphoethanolamine-N-(hexanoylamine), 1,2-dioleoyl-sn-glycero-3-phosphoethanolamine-N-(hexanoylamine), 1,2-dipalmitoyl-sn-glycero-3-phosphoethanolamine-N-[4-(p-maleimidophenyl)butyramide], 1,2-dioleoyl-sn-glycero-3-phosphoethanolamine-N-[4-(p-maleimidophenyl)butyramide], 1,2-dipalmitoyl-sn-glycero-3-phospho(ethylene glycol), 1,2-Dioleoyl-sn-Glycero-3-Phospho(Ethylene Glycol), 1,2-dipalmitoyl-sn-glycero-3-phosphoethanolamine-N-(6-((folate)amino)hexanoyl), 1,2-dipalmitoyl-sn-glycero-3-phosphoethanolamine-N-(cyanur), 1,2-dipalmitoyl-sn-glycero-3-phosphoethanolamine-N-(biotinyl), 1,2-dioleoyl-sn-glycero-3-phosphoethanolamine-N-(biotinyl), 1,2-dipalmitoyl-sn-glycero-3-phosphoethanolamine-N-(cap biotinyl), 1,2-dipalmitoyl-sn-glycero-3-phosphoethanolamine-N-{6-[(cyanur)amino]hexanoyl}, 1,2-dioleoyl-sn-glycero-3-phosphoethanolamine-N-(cap biotinyl), 1,2-dipalmitoyl-sn-glycero-3-phosphoethanolamine-N-(dodecanoyl), 1,2-dioleoyl-sn-glycero-3-phosphoethanolamine-N-(dodecanyl), 1,2-dipalmitoyl-sn-glycero-3-phosphoethanolamine-N-(glutaryl), 1,2-dioleoyl-sn-glycero-3-phosphoethanolamine-N-(glutaryl), 1,2-dipalmitoyl-sn-glycero-3-phosphoethanolamine-N-(succinyl), 1,2-dioleoyl-sn-glycero-3-phosphoethanolamine-N-(succinyl), 1,2-dipalmitoyl-sn-glycero-3-phosphoethanolamine-N-[3-(2-pyridyldithio)propionate], 1,2-dioleoyl-sn-glycero-3-phosphoethanolamine-N-[3-(2-pyridyldithio)propionate], 1,2-Dipalmitoyl-sn-Glycero-3-Phosphothioethanol, 1,2-dioleoyl-sn-glycero-3-phosphoethanolamine-N-(dodecanylamine), 1,2-dipalmitoyl-sn-glycero-3-phosphoethanolamine-N-(dodecanylamine), 1,2-dipalmitoyl-sn-glycero-3-phosphoethanolamine-N-[4-(p-maleimidomethyl)cyclohexane-carboxamide], 1,2-dioleoyl-sn-glycero-3-phosphoethanolamine-N-[4-(p-maleimidomethyl)cyclohexane-carboxamide], 1,2-dipalmitoyl-sn-glycero-3-phosphoethanolamine-N-(5-hexynoyl), 1,2-dipalmitoyl-sn-glycero-3-phosphoethanolamine-N-(6-azidohexanoyl), 1,2-distearoyl-sn-glycero-3-phosphoethanolamine-N-(maleimide), 1,2-distearoyl-sn-glycero-3-phosphoethanolamine-n-(dib enzocycooctyl), 1,2-distearoyl-sn-glycero-3-phosphoethanolamine-N-[10-(trimethoxysilyl)undecanamide], 1,2-distearoyl-sn-glycero-3-phosphoethanolamine-N—(PDP), 1,2-distearoyl-sn-glycero-3-phosphoethanolamine-N-(carboxy), 1,2-distearoyl-sn-glycero-3-phosphoethanolamine-N-(folate), and N-(4-carb oxybenzyl)-N,N-dimethyl-2,3-bis(oleoyloxy)propan-1-aminium. In some embodiments, the functionalized lipid is 1,2-distearoyl-sn-glycero-3-phosphoethanolamine-N-[carboxy(polyethylene glycol)-2000]-COOH (DSPE-PEG-COOH).

In some embodiments, the non-cationic liposomes of the present disclosure comprises neutral lipid (e.g., DOPC), a pH-responsive lipid (e.g., DODAP), and a functionalized lipid (DSPE-PEG-COOH). In some embodiments, the neutral lipid is 50%-99% (e.g., by molar ratio or by weight) of the total lipid composition of the lipid bilayer. For example, the neutral lipid may be 50%-99%, 50%-95%, 50%-90%, 50%-85%, 50%-80%, 50%-75%, 50%-70%, 50%-65%, 50%-60%, 50%-55%, 55%-99%, 55%-95%, 55%-90%, 55%-85%, 55%-80%, 55%-75%, 55%-70%, 55%-65%, 55%-60%, 60%-99%, 60%-95%, 60%-90%, 60%-85%, 60%-80%, 60%-75%, 60%-70%, 60%-65%, 65%-99%, 65%-95%, 65%-90%, 65%-85%, 65%-80%, 65%-75%, 65%-70%, 70%-99%, 70%-95%, 70%-90%, 70%-85%, 70%-80%, 70%-75%, 75%-99%, 75%-95%, 75%-90%, 75%-85%, 75%-80%, 80%-99%, 80%-95%, 80%-90%, 80%-88%, 85%-99%, 85%-95%, 85%-90%, 90%-99%, 90%-95%, or 95%-99% (e.g., by molar ratio or by weight) of the total lipid composition of the lipid bilayer. In some embodiments, the neutral lipid is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% (e.g., by molar ratio or by weight) of the total lipid composition of the lipid bilayer.

In some embodiments, the pH-responsive lipid is 1%-40% (e.g., by molar ratio or by weight) of the total lipid composition of the lipid bilayer. For example, the pH-responsive lipid may be 1%-40%, 1%-35%, 1%-30%, 1%-25%, 1%-20%, 1%-15%, 1%-10%, 1%-5%, 5%-40%, 5%-35%, 5%-30%, 5%-25%, 5%-20%, 5%-15%, 5%-10%, 10%-40%, 10%-35%, 10%-30%, 10%-25%, 10%-20%, 10%-15%, 15%-40%, 15%-35%, 15%-30%, 15%-25%, 15%-20%, 20%-40%, 20%-35%, 20%-30%, 20%-25%, 25%-40%, 25%-35%, 25%-30%, 30%-40%, 30%-35%, or 35%-40% (e.g., by molar ratio or by weight) of the total lipid composition of the lipid bilayer. In some embodiments, the pH-responsive lipid is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% (e.g., by molar ratio or by weight) of the total lipid composition of the lipid bilayer. In some embodiments, the lipid bilayer of the liposome does not contain a pH-responsive lipid (i.e., 0% by molar ratio or by weight).

In some embodiments, the functionalized lipid is 1%-20% (e.g., by molar ratio or by weight) of the total lipid composition of the lipid bilayer. For example, the functionalized lipid may be 1%-20%, 1%-15%, 1%-10%, 1%-5%, 5%-20%, 5%-15%, 5%-10%, 10%-20%, 10%-15%, or 15%-20% (e.g., by molar ratio or by weight) of the total lipid composition of the lipid bilayer. In some embodiments, the functionalized lipid is 1%, 2%, 3%, 4%, 5%, 6%, 7%, 8%, 9%, 10%, 11%, 12%, 13%, 14%, 15%, 16%, 17%, 18%, 19%, or 20% (e.g., by molar ratio or by weight) of the total lipid composition of the lipid bilayer. In some embodiments, the functionalized lipid is up to 10% (e.g., 10%, 9%, 8%, 7%, 6%, 5%, 4%, 3%, 2%, or 1%) the total lipid composition of the lipid bilayer. In some embodiments, higher (e.g., more than 20%) or lower (e.g., less than 1%) percentages of functionalized lipid in the lipid bilayer is also contemplated. The percentage of the functionalized lipid is at least in part related to the amount of ligands needed to be conjugated to the liposome containing the functionalized lipids.

In some embodiments, the molar ratio of the neutral lipid, the pH-responsive lipid, and the functionalized lipid in the lipid bilayer of the liposomes described herein is 65%:30%:5%. In some embodiments, the molar ratio of the neutral lipid, the pH-responsive lipid, and the functionalized lipid in the lipid bilayer of the liposomes described herein is 85%:10%:5%.

Liposomes containing functionalized lipids may be referred to as functionalized liposomes. The functional groups of the functional lipids are arranged on the outer surface of the liposome, allowing attaching or conjugation of a wide range of molecules (e.g., nucleic acids, polypeptides or proteins, organic compounds, etc.) to the surface of the functionalized liposomes. In some embodiments, the molecule is a ligand.

A “ligand,” as used herein, refers to a molecule that specifically binds to and forms a complex with another molecule (e.g., a biomolecule such as a protein). The molecule that is bound by the ligand is herein referred as a “target molecule.” In some embodiments, the target molecule is a protein, e.g., a receptor protein. In some embodiments, the target molecular is a cell surface receptor protein. The binding of a ligand to its target molecule may be via intermolecular forces, such as ionic bonds, hydrogen bonds and Van der Waals forces. In some embodiments, the binding of a ligand to its target molecule (e.g., a receptor protein) serves a biological purpose. For example, binding of a ligand to a receptor protein alters the chemical conformation by affecting the three-dimensional shape orientation. The conformation of a receptor protein composes its functional state. Ligands include substrates, inhibitors, activators, antibodies, and neurotransmitters. The rate of binding is called affinity (KD), and this measurement typifies a tendency or strength of the effect of binding. Binding affinity is actualized not only by host-guest interactions, but also by solvent effects that can play a dominant, steric role which drives non-covalent binding in solution. The solvent provides a chemical environment for the ligand and receptor to adapt, and thus accept or reject each other as partners.

The term “bind” refers to the association of two entities (e.g., two proteins). Two entities (e.g., two proteins) are considered to bind to each other when the affinity (KD) between <10⁻³ M, <10⁻⁴ M, <10⁻⁵ M, <10⁻⁶ M, <10⁻⁷ M, <10⁻⁸ M, <10⁻⁹ M, <10⁻¹⁰ M, <10⁻¹¹ M, or <10⁻¹² M. One skilled in the art is familiar with how to assess the affinity of two entities (e.g., two proteins).

Any ligands (e.g., a protein ligand) may be conjugated to the surface of the liposomes described herein. The terms conjugating, conjugated, and conjugation refer to an association of two entities, for example, of two molecules (e.g., two proteins), two domains, or a protein and an agent, e.g., a protein and a lipid. The association can be, for example, via a direct or indirect (e.g., via a linker) covalent linkage or via non-covalent interactions. In some embodiments, the association is covalent. For example, in some embodiments, the a protein and a lipid is conjugated via the reactive group on a functionalized lipid, the association between the protein and the lipid is covalent. In some embodiments, two molecules are conjugated via a linker connecting both molecules.

In some embodiments, a ligand (e.g., a protein ligand) may be conjugated to the surface of the liposome via the functional group on the functionalized lipid in the liposome. For example, without limitation, a functionalized lipid containing carboxylic acid group may react with the amine group at the N-terminus of a protein or polypeptide ligand, thereby conjugating the protein or polypeptide ligand to the surface of the liposome. Methods of conjugating a ligand via a reactive or functional group is known to those skilled in the art.

In some embodiments, the ligand of the present disclosure targets ICAM-1 (ICAM-1 ligands). In some embodiments, the ligand of the present disclosure targets EGFR (EGFR ligands). In some embodiments, the nanoparticles of the present disclosure comprises a first ligand targeting ICAM-1 and a second ligand targeting EGFR conjugated to its surface. Nanoparticles comprising ligands targeting other cell surface proteins are also within the scope of the present disclosure.

“Intercellular adhesion molecule 1” or “ICAM-1” is a member of the super-immunoglobulin family of molecules. Members of this superfamily are characterized by the presence of one or more Ig homology regions, each consisting of a disulfide-bridged loop that has a number of anti-parallel β-pleated strands arranged in two sheets. Three types of homology regions have been defined, each with a typical length and having a consensus sequence of amino acid residues located between the cysteines of the disulfide bond. (Williams, A. F. et al., Ann. Rev. Immunol. 6:381-405 (1988); Hunkapillar, T. et al., Adv. Immunol. 44:1-63 (1989)). ICAM-1 is a cell surface glycoprotein of 97-114 kd. ICAM-1 has 5 Ig-like domains. Its structure is closely related to those of the neural cell adhesion molecule (NCAM) and the myelin-associated glycoprotein (MAG) (e.g., as described Simmons, D. et al., Nature 331:624-627 (1988); Staunton, D. E. et al., Cell 52:925-933 (1988); Staunton, D. E. et al., Cell 61243-254 (1990), herein incorporated by reference). ICAM has previously been shown to overexpression on TNBC cells and has been characterized as a molecular target for TNBC (e.g., as described in Guo et al., PNAS, vol. 111, no. 41, pages 14710-14715, 2014; and Guo et al., Theranostics, Vol. 6, Issue 1, 2016, incorporated herein by reference).

“Epidermal growth factor receptor” or “EGFR” is the cell-surface receptor for members of the epidermal growth factor family (EGF family) of extracellular protein ligands. Mutations that lead to EGFR overexpression (also known as upregulation) or overactivity have been associated with a number of cancers, including squamous-cell carcinoma of the lung (about 80% of cases), anal cancers, glioblastoma (about 50%) and epithelial tumors of the head and neck (about 80-100%). These somatic mutations involving EGFR lead to its constant activation, which produces uncontrolled cell division.

Suitable ligands that may be conjugated to the non-cationic liposomes include, without limitation: antibodies or antibody fragments, inhibitory peptides including peptides derived from natural proteins and synthetic peptides, natural inhibitory ligands, small molecules (e.g., small molecule inhibitors), and aptamers.

EGFR and ICAM-1 have been shown to overexpress on cancer cells (e.g., triple negative breast cancer cells) and therefor may be targeted by the ligands conjugated to the surface of the liposomes. The EGFR ligands described herein do not encompass natural EGFR ligands that activate EGFR signaling, e.g., TGF-α and EGF. In some embodiments, an EGFR ligand binds to EGFR on the surface of a cancer/tumor cell. The ICAM-1 ligands described herein bind to ICAM-1 on the surface of a cancer/tumor cell. In some embodiments, the ICAM-1 ligands of the present disclosure blocks/inhibits ICAM-1 signaling in the tumor cell, leading to inhibition of tumor growth. The EGFR ligands of the present disclosure blocks/inhibits the interaction between EGFR and its activating ligands. In some embodiments, the binding of the EGFR ligand to EGFR blocks/inhibits EGFR signaling in the tumor cell, leading to inhibition of tumor growth.

“Antibodies” and “antibody fragments” include whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. An antibody may be a polyclonal antibody or a monoclonal antibody.

An “antibody fragment” for use in accordance with the present disclosure contains the antigen-binding portion of an antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

EGFR antibodies that inhibit EGFR signaling are known in the art and have been used for treatment of cancer, e.g., without limitation, Erbitux (generic name: cetuximab), Vectibix (generic name: panitumumab), Portrazza (generic name: necitumumab). ICAM-1 antibodies are known to those skilled in the art and are commercially available (e.g., from Santa Cruz or Abcam).

“Inhibitory peptides” refers to peptides that specifically binds to a target molecule. In some embodiments, binding of an inhibitory peptide to a target molecule inhibits the biological activity of the target molecule. For example, if the target molecule functions in a signaling pathway, binding of the inhibitory peptide may inhibit the signaling pathway. One skilled in the art is familiar with inhibitory peptides or methods of developing inhibitory peptides to their target molecule of choice. For example, peptides that are derived from the EGFR-binding portion of proteins that binds to EGFR (e.g., epidermal growth factor or EGF) may be used as an inhibitory peptide in accordance with the present disclosure. An inhibitory peptides may also be synthetic (i.e., synthetic peptides). Similarly, peptides that are derived from the ICAM-1 binding portion of proteins that binds to ICAM-1 (e.g., integrin) may be used as an inhibitory peptide in accordance with the present disclosure. Synthetic peptides may be obtained using methods that are known to those skilled in the art. Synthetic peptides that inhibit EGFR signaling are known in the art, e.g., as described in Ahsan et al., Neoplasia, Volume 16, Issue 2, February 2014, Pages 105-114; and in Sinclair et al., Org Lett. 2014 Sep. 19; 16(18):4916-9, incorporated herein by reference. Synthetic peptides that inhibit ICAM-1 function are known in the art, e.g., as described in Zimmerman et al., Chem Biol Drug Des. 2007 October; 70(4):347-53. Epub 2007, incorporated herein by reference.

An “aptamer” refers to an oligonucleotide or a peptide molecule that binds to a specific target molecule. Aptamers are usually created by selecting them from a large random sequence pool. Aptamers that inhibit EGFR signaling are known to those skilled in the art, e.g., as described in Li et al., PloS ONE, Volume 6, Issue 6, e20299, 2011, Liu et al., Biol Chem. 2009 February; 390(2): 10.1515/BC.2009.022, and US Patent Application Publication US20130177556, incorporated herein by reference.

A “natural ligand” is a ligand that exists in nature. The present disclosure encompass natural ligands for proteins that specifically express or overexpress on the surface of a cell targeted by the nanoparticles described herein (e.g., a cancer cell).

A “lipid” refers to a group of naturally occurring molecules that include fats, waxes, sterols, fat-soluble vitamins (such as vitamins A, D, E, and K), monoglycerides, diglycerides, triglycerides, phospholipids, and others. A “monosaccharide” refers to a class of sugars (e.g., glucose) that cannot be hydrolyzed to give a simpler sugar. Non-limiting examples of monosaccharides include glucose (dextrose), fructose (levulose) and galactose. A “second messenger” is a molecule that relay signals received at receptors on the cell surface (e.g., from protein hormones, growth factors, etc.) to target molecules in the cytosol and/or nucleus. Non-limiting examples of second messenger molecules include cyclic AMP, cyclic GMP, inositol trisphosphate, diacylglycerol, and calcium. A “metabolite” is an molecule that forms as an intermediate produce of metabolism. Non-limiting examples of a metabolite include ethanol, glutamic acid, aspartic acid, 5′ guanylic acid, Isoascorbic acid, acetic acid, lactic acid, glycerol, and vitamin B2. A “xenobiotic” is a foreign chemical substance found within an organism that is not normally naturally produced by or expected to be present within. Non-limiting examples of xenobiotics include drugs, antibiotics, carcinogens, environmental pollutants, food additives, hydrocarbons, and pesticides.

A “small molecule,” as used herein, refers to a molecule of low molecular weight (e.g., <900 daltons) organic or inorganic compound that may function in regulating a biological process. Non-limiting examples of a small molecule include lipids, monosaccharides, second messengers, other natural products and metabolites, as well as drugs and other xenobiotics.

Small molecule inhibitors of EGFR and ICAM-1 are also known to those skilled in the art. Non-limiting, exemplary small molecule inhibitors for EGFR include AEE 788, AG 1478 hydrochloride, AG 18, AG 490, AG 494, AG 555, AG 556, AG 825, AG 879, AG 99, AV 412 New product, BIBU 1361 hydrochloride, BMX 1382 dihydrochloride, BMS 599626 dihydrochloride, Canertinib dihydrochloride, CGP 52411, CP 724714, DIM, Genistein, GW 583340 dihydrochloride, HDS 029, HKI 357, Iressa, JNJ 28871063 hydrochloride, Lavendustin A, Methyl 2,5-dihydroxycinnamate, PD 153035 hydrochloride, PD 158780, PF 6274484, PKI 166 hydrochloride, PP 3, TAK 165, Tyrphostin B44, (−) enantiomer, Tyrphostin B44, (+) enantiomer, and WHI-P 154. Non-limiting, exemplary small molecule inhibitors for EGFR include metadichol, methimazole, and silibinin.

Multiple ligands may be conjugated to the surface of the non-cationic liposome of the present disclosure, each ligand targeting a different cell surface protein. In some embodiments, 2-10 cell surface proteins are targeted by the ligands conjugated to the surface of the liposome. For example, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 cell surface proteins are targeted. In some embodiments, 1, 2, 3, 4, 5, 6, 7, 8, 9, or 10 cell surface proteins are targeted.

In some embodiments, the non-cationic liposome described herein may be engineered such that it specifically targets one cell type (e.g., a cancer cell) but no other cell types (e.g., a normal cell). As such, the ligands conjugated to the surface of the non-cationic liposome are ligands that binds to cell surface proteins that specifically express or overexpress on one cell type cell type (e.g., a cancer cell) but not in other cell types (e.g., a normal cell). Surface proteins that specifically express or overexpress on one cell type but not in other cell types may be identified by any known methods in the art, e.g., western blotting, immunostaining, flow-cytometry or mass-spectrometry. One skilled in the art is familiar with how to identify target proteins on the surface of the target cell, and choose appropriate ligands that binds the target protein.

A protein (e.g., membrane protein) that specifically expresses on the surface of one cell type but not another refers to a protein that is only detectable on one cell type using any protein detection methods known in the art (e.g., western blotting, immunostaining, flow-cytometry or mass-spectrometry), but is not detectable on any other cell types. A protein that overexpresses on the surface of one cell type compared to another refers to a protein whose surface expression level is higher than that of another cell type. For example, the expression level of an overexpressed protein on the surface of one cell type may be at least 20% higher than its expression level on the surface of another cell type. In some embodiments, the expression level of an overexpressed protein on the surface of one cell type is at least 20%, at least 30%, at least 40%, at least 50%, at least 60%, at least 70%, at least 80%, at least 90%, at least 100%, at least 2-fold, at least 3-fold, at least 4-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, or at least 1000-fold higher than its expression level on the surface of another cell type. In some embodiments, the expression level of an overexpressed protein on the surface of one cell type is 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 60-fold, 70-fold, 80-fold, 90-fold, 100-fold, or 1000-fold higher than its expression level on the surface of another cell type. In some embodiments, the expression level of an overexpressed protein on the surface of one cell type is more than 1000-fold higher than its expression level on the surface of another cell type. In some embodiments, a protein that overexpresses on the surface of a cell may also be overexpressed in the cell (i.e., intracellularly). In some embodiments, a protein that overexpresses on the surface of a cell is not overexpressed in the cell.

The nanoparticles of the present disclosure further comprises a hydrogel encapsulated in the non-cationic liposome. “Encapsulated” means the therapeutic agent is enclosed in the aqueous volume created by the completely closed lipid bilayer of the liposome. “Hydrogel” refers to a water-swellable polymeric matrix formed from a three-dimensional network of macromolecules held together by covalent or non-covalent crosslinks, that can absorb a substantial amount of water (by weight) to form a gel. Liposomes with a hydrgel core have enhanced encapsulation efficiency and ratio of the agents to be encapsulated in the liposome. “Having enhanced encapsulation efficiency and ratio” means that a liposome with a hydrogel core is able to encapsulate more agents (e.g., at least 20%, at least 30%, at least 40%, at least 50%, at least 60%, at least 70%, at least 80%, at least 90%, at least 100%, at least 2-fold, at least 3-fold, at least 4-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 1000-fold or more), relative to a liposome without a hydrogel core.

In some embodiments, the hydrogel comprises crosslinked block copolymer containing one or more poly(alkylene oxide) segments, such as polyethylene glycol, and one or more aliphatic polyester segments, such as polylactic acid. One or more host molecules, such as a cyclodextrin, dendrimer, or ion exchange resin, is dispersed within or covalently bound to the polymeric matrix.

In some embodiments, the hydrogel may be formed from one or more polymers or copolymers. The polymers may be synthetic or naturally occurring. Non-limiting, exemplary polymers include: poly(lactic acid), poly(glycolic acid), poly(lactic acid-co-glycolic acids), polyhydroxyalkanoates such as poly3-hydroxybutyiate or poly4-hydroxybutyrate; polycaprolactones; poly(orthoesters); polyanhydrides; poly(phosphazenes); poly(lactide-co-caprolactones); poly(glycolide-co-caprolactones); polycarbonates such as tyrosine polycarbonates; polyamides (including synthetic and natural polyamides), polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides) such as polyethylene glycol (PEG); derivativized celluloses such as alkyl celluloses (e.g., methyl cellulose), hydroxyalkyl celluloses (e.g., hydroxypropyl cellulose), cellulose ethers, cellulose esters, nitrocelluloses, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl aciylate) (jointly referred to herein as “polyacrylic acids”), as well as derivatives, copolymers, and combinations thereof.

In some embodiments, derivatives of polymers are used in the hydrogel. “Derivatives” include polymers having substitutions, additions of chemical groups and other modifications to the polymeric backbones described above routinely made by those skilled in the art. Natural polymers, including proteins such as albumin, collagen, gelatin, prolamines, such as zein, and polysaccharides such as alginate and pectin, may also be incorporated into the polymeric matrix. In certain cases, when the polymeric matrix contains a natural polymer, the natural polymer is a biopolymer which degrades by hydrolysis, such as a polyhydroxyalkanoate.

In some embodiments, the hydrogel comprises one or more cross linkable polymers. In some embodiments, the cross linkable polymers contain one or more photo-polymerizable groups, allowing for the crosslinking of the polymeric matrix following nanolipogel formation. Examples of suitable photo-polymerizable groups include, without limitation, vinyl groups, acrylate groups, methacrylate groups, and acrylamide groups. Photo-polymerizable groups, when present, may be incorporated within the backbone of the cross linkable polymers, within one or more of the sidechains of the cross linkable polymers, at one or more of the ends of the crosslinkable polymers, or combinations thereof.

In some embodiments, the hydrogel is formed from a poly(alkylene oxide) polymer or a block copolymer containing one or more poly(alkylene oxide) segments. The poly(alkylene oxide) polymer or poly(alkylene oxide) polymer segments may contain between 8 and 500 repeat units, between 40 and 300 repeat units, or between 50 and 150 repeat units. Suitable poly(alkylene oxides) include polyethylene glycol (also referred to as polyethylene oxide or PEG), polypropylene 1,2-glycol, polypropylene oxide), polypropylene 1,3-glycol, and copolymers thereof.

In some embodiments, the hydrogel comprises an aliphatic polyester or a block copolymer containing one or more aliphatic polyester segments. In some embodiments, the polyester or polyester segments are poly(lactic acid) (PLA), poly(glycolic acid) PGA, or poly(lactide-co-glycolide) (PLGA). In some embodiments, the hydrogel comprises a block copolymer containing one or more poly(alkylene oxide) segments, one or more aliphatic polyester segments, and optionally one or more photo-polymerizable groups.

In some embodiments, the hydrogel comprises a material selected from the group consisting of: alginate, alginate derivatives, albumin, collagen, gelatin, prolamines, polysaccharides, chitosan, metrigel, polylysine, alginic acid, carrageenan, chondroitin sulfate, dextran sulfate, pectin, carboxymethyl chitin, fibrin, agarose, dextran, pullulan, poly(vinylsulfonic acid), poly(2-suloethylmethacrylate), poly(2-sulfoethyl acrylate), poly(2-(dimethylamino)ethyl methacrylate), poly(2-(dimethylamino)ethyl acrylate), poly(2-(di ethylamino)ethyl acrylate), poly(lactic acid), poly(glycolic acid), poly(lactic acid-co-glycolic acids), polyhydroxyalkanoates, polycaprolactones, poly(orthoesters), polyanhydrides, poly(phosphazenes), poly(lactide-co-caprolactones), poly(glycolide-co-caprolactones), polycarbonates, polyamides, polypeptides, poly(amino acids), polyesteramides, polyesters, poly(dioxanones), poly(alkylene alkylates), hydrophilic polyethers, polyurethanes, polyetheresters, polyacetals, polycyanoacrylates, polysiloxanes, poly(oxyethylene)/poly(oxypropylene) copolymers, polyketals, polyphosphates, polyhydroxyvalerates, polyalkylene oxalates, polyalkylene succinates, poly(maleic acids), polyvinyl alcohols, polyvinylpyrrolidone, poly(alkylene oxides), celluloses, polyacrylic acids, derivatives, copolymers, and combinations thereof.

In some embodiments, the hydrogel comprises an alginate (e.g., sodium alginate). Methods of producing a nanoparticle comprising a sodium alginate hydrogel core are known in the art, e.g., an extrusion method as described in U.S. Pat. No. 5,626,870, incorporated herein by reference. In such methods, the lipids for making the liposome are mixed and dissolved in a solvent and dried to form a lipid film. The lipid film is then hydrated in a sodium alginate solution and extruded through a nanoporous membrane with specific a pore size. The resulting nanoparticle contains the hydrogel core and typically has a diameter of more than 200 nm, and has a broad size distribution.

The nanoparticle of the present disclosure, in some embodiments, has a diameter of less than 200 nm. For example, the nanoparticle of the present disclosure may have a diameter of no more than 200 nm, no more than 190 nm, no more than 180 nm, no more than 170 nm, no more than 160 nm, no more than 150 nm, no more than 140 nm, no more than 130 nm, no more than 120 nm, no more than 110 nm, no more than 100 nm, or less. In some embodiments, nanoparticle of the present disclosure has a diameter of 100 nm, 110 nm, 120 nm, 130 nm, 140 nm, 150 nm, 160 nm, 170 nm, 180 nm, 190 nm, or 200 nm.

It is difficult to produce uniform and monodisperse nanoparticles of less than 200 nm using the traditional extrusion methods, because it is difficult to be directly extrude a lipid/hydrogel solution that has not previously been extruded through a membrane with larger pores (e.g., 400 nm) through a nanoporous membrane with a pore size of 100 or 200 nm. The methods developed in the present disclosure enables the generation of uniform and monodisperse nanoparticles with a diameter of no more than 200 nm. Herein, the lipids for making a liposome (e.g., the neutral lipid, the pH-responsive lipid, and the functionalized lipid) are dissolved in a solvent (e.g., chloroform) and dried to form a lipid film. The lipid film is then hydrated in a sodium alginate solution (e.g., at a concentration of 1 mg/ml) and extruded through a series of nanoporous membranes (e.g., polycarbonate track-etched membranes) with pore sizes in the order of 400, 200, and 100 nm. The series extrusion steps enable the generation of monodisperse nanoparticles having a diameter of no more 200 nm.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which at least 90% (e.g., 90%, 91%, 92%, 93%, 94%, 95%, 96%, 97%, 98%, 99%, or 100%) of the distribution lies within 15% (e.g., 15%, 14%, 13%, 12%, 11%, 10%, 9%, 8%, 7%, 6%, 5%, 4%, 3%, 2%, 1%, or less) of the median particle size, or the same as the median particle size.

The nanoparticle of the present disclosure may be used as a delivery system to deliver an agent into a cell. A “delivery system,” as used herein, refers to a system (e.g., the nanoparticle described herein) that may be used to deliver an agent across the cell membrane into the cytoplasm of the cell. Thus, in some embodiments, the nanoparticles of the present disclosure further comprises an agents encapsulated in the non-cationic liposome. The liposome drug delivery system may be designed to target any cell where delivery of the therapeutic agent is desired. One skilled in the art is able to ascertain the cell type and choose appropriate pharmaceutically compositions.

The “agent” encapsulated in the non-cationic liposome may be a physiologically or pharmacologically active substance that acts locally and/or systemically in the body. The agent may be used for the treatment (e.g., therapeutic agent), prevention (e.g., prophylactic agent), or diagnosis (e.g., diagnostic agent) of a disease or disorder. A “therapeutic agent” is an agent that has therapeutic effects on, and may be used to treat any diseases or conditions. A therapeutic agent may be a small molecule, an oligonucleotide, a polypeptide or a protein, and combinations thereof.

In some embodiments, the therapeutic agent is an anti-cancer agent. An “anti-cancer agent” is any agent that is able to inhibit growth of and/or kills cancer cells, and/or prevent metastasis. In some embodiments, an anti-cancer agent is a chemotherapeutic agent. A “chemotherapeutic agent” is a chemical agent or drugs that are selectively destructive to malignant cells and tissues. Non-limiting, exemplary chemopharmaceutically compositions that may be used in the liposome drug delivery systems of the present disclosure include, Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine. In some embodiments, the chemotherapeutic agent is Doxorubicin.

In some embodiments, the anticancer agent is an oligonucleotide (e.g., an siRNA, shRNA, or miRNA targeting an oncogene). An “oncogene” is a gene that in certain circumstances can transform a cell into a tumor cell. An oncogene may be a gene encoding a growth factor or mitogen (e.g., c-Sis), a receptor tyrosine kinase (e.g., EGFR, PDGFR, VEGFR, or HER2/neu), a cytoplasmic tyrosine kinase (e.g., Src family kinases, Syk-ZAP-70 family kinases, or BTK family kinases), a cytoplasmic serine/threonine kinase or their regulatory subunits (e.g., Raf kinase or cyclin-dependent kinase), a regulatory GTPase (e.g., Ras), or a transcription factor (e.g., Myc). In some embodiments, the oligonucleotide targets Lipocalin (Lcn2) (e.g., a Lcn2 siRNA). One skilled in the art is familiar with genes that may be targeted for the treatment of cancer.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. In some embodiments, the anticancer agent is a protein or polypeptide-based anti-cancer agent, e.g., an antibody. Anti-cancer antibodies are known to those skilled in the art.

Non-limiting, exemplary protein or polypeptide-based therapeutic agents include enzymes, regulatory proteins (e.g., immuno-regulatory proteins), antigens, antibodies or antibody fragments, and structural proteins. In some embodiments, the protein or polypeptide-based therapeutic agents are for cancer therapy.

Suitable enzymes for some embodiments of this disclosure include, for example, oxidoreductases, transferases, polymerases, hydrolases, lyases, synthases, isomerases, and ligases, digestive enzymes (e.g., proteases, lipases, carbohydrases, and nucleases). In some embodiments, the enzyme is selected from the group consisting of lactase, beta-galactosidase, a pancreatic enzyme, an oil-degrading enzyme, mucinase, cellulase, isomaltase, alginase, digestive lipases (e.g., lingual lipase, pancreatic lipase, phospholipase), amylases, cellulases, lysozyme, proteases (e.g., pepsin, trypsin, chymotrypsin, carboxypeptidase, elastase), esterases (e.g. sterol esterase), disaccharidases (e.g., sucrase, lactase, beta-galactosidase, maltase, isomaltase), DNases, and RNases.

Non-limiting, exemplary antibodies and fragments thereof include: bevacizumab (AVASTIN®), trastuzumab (HERCEPTIN®), alemtuzumab (CAMPATH®, indicated for B cell chronic lymphocytic leukemia), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-05, IOR-T6 (anti-CD1), IOR EGF/R3, celogovab (ONCOSCINT® OV103), epratuzumab (LYMPHOCIDE®), pemtumomab (THERAGYN®) and Gliomab-H (indicated for brain cancer, melanoma). Other antibodies and antibody fragments are contemplated and may be used in accordance with the disclosure.

A regulatory protein may be, in some embodiments, a transcription factor or a immunoregulatory protein. Non-limiting, exemplary transcriptional factors include: those of the NFkB family, such as Rel-A, c-Rel, Rel-B, p50 and p52; those of the AP-1 family, such as Fos, FosB, Fra-1, Fra-2, Jun, JunB and JunD; ATF; CREB; STAT-1, -2, -3, -4, -5 and -6; NFAT-1, -2 and -4; MAF; Thyroid Factor; IRF; Oct-1 and -2; NF-Y; Egr-1; and USF-43, EGR1, Sp 1, and E2F1.

As used herein, an immunoregulatory protein is a protein that regulates an immune response. Non-limiting examples of immunoregulatory include: antigens, adjuvants (e.g., flagellin, muramyl dipeptide), cytokines including interleukins (e.g., IL-2, IL-7, IL-15 or superagonist/mutant forms of these cytokines), IL-12, IFN-gamma, IFN-alpha, GM-CSF, FLT3-ligand), and immunostimulatory antibodies (e.g., anti-CTLA-4, anti-CD28, anti-CD3, or single chain/antibody fragments of these molecules). Other immunostimulatory proteins are contemplated and may be used in accordance with the disclosure.

As used herein, an antigen is a molecule or part of a molecule that is bound by the antigen-binding site of an antibody. In some embodiments, an antigen is a molecule or moiety that, when administered to or expression in the cells of a subject, activates or increases the production of antibodies that specifically bind the antigen. Antigens of pathogens are well known to those of skill in the art and include, but are not limited to parts (coats, capsules, cell walls, flagella, fimbriae, and toxins) of bacteria, viruses, and other microorganisms. Examples of antigens that may be used in accordance with the disclosure include, without limitation, cancer antigens, self-antigens, microbial antigens, allergens and environmental antigens.

In some embodiments, the antigen of the present disclosure is a cancer antigen. A cancer antigen is an antigen that is expressed preferentially by cancer cells (i.e., it is expressed at higher levels in cancer cells than on non-cancer cells) and, in some instances, it is expressed solely by cancer cells. Cancer antigens may be expressed within a cancer cell or on the surface of the cancer cell. Cancer antigens that may be used in accordance with the disclosure include, without limitation, MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4 and MAGE-05. The cancer antigen may be selected from the group consisting of GAGE-1, GAGE-2, GAGE-3, GAGE-4, GAGE-5, GAGE-6, GAGE-7, GAGE-8 and GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pme1117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20 and c-erbB-2. Other cancer antigens are contemplated and may be used in accordance with the disclosure.

In some embodiments, the agent encapsulated in the nanoparticles described herein is a genome-editing agent. The term “genome” refers to the genetic material of a cell or organism. It typically includes DNA (or RNA in the case of RNA viruses). The genome includes both the genes, the coding regions, the noncoding DNA, and the genomes of the mitochondria and chloroplasts. A genome does not typically include genetic material that is artificially introduced into a cell or organism, e.g., a plasmid that is transformed into a bacteria is not a part of the bacterial genome. A “genome-editing agent” refers to an agent that is capable of inserting, deleting, or replacing nucleotide(s) in the genome of a living organism. In some embodiments, a genome editing agent is an engineered nuclease that can create site-specific double-strand breaks (DSBs) at desired locations in the genome. The induced double-strand breaks are repaired through nonhomologous end-joining (NHEJ) or homologous recombination (HR), resulting in targeted mutations (‘edits’). As such, the engineered nucleases suitable for genome-editing may be programmed to target any desired sequence in the genome and are also referred to herein as “programmable nucleases.” Suitable programmable nucleases for genome-editing that may be used in accordance with the present disclosure include, without limitation, meganucleases, zinc finger nucleases (ZFNs), transcription activator-like effector-based nucleases (TALEN), and the CRISPR/Cas system. One skilled in the art is familiar with the programmable nucleases and methods of using them for genome-editing. For example, methods of using ZFNs and TALENs for genome-editing are described in Maeder, et al., Mol. Cell 31 (2): 294-301, 2008; Carroll et al., Genetics Society of America, 188 (4): 773-782, 2011; Miller et al., Nature Biotechnology 25 (7): 778-785, 2007; Christian et al., Genetics 186 (2): 757-61, 2008; Li et al., Nucleic Acids Res 39 (1): 359-372, 2010; and Moscou et al., Science 326 (5959): 1501, 2009, incorporated herein by reference.

In some embodiments, the genome-editing agent is a Clustered regularly interspaced short palindromic repeats (CRISPR)/Cas system. A “CRISPR/Cas system” refers to a prokaryotic adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), incorporated herein by reference.

Cas9 orthologs have been described in various species, including, but not limited to, S. pyogenes (e.g., as described in Jinek et al., Science 337:816-821(2012), incorporated herein by reference); and Cpf1 (CRISPR from Prevotella and Francisella 1 (e.g., as described in Zetsche et al., Cell, 163, 759-771, 2015, incorporated herein by reference).

Cas9 and Cpf1 nuclease sequences and structures are well known to those of skill in the art (see, e.g., Ferretti et al., Proc. Natl. Acad. Sci. 98:4658-4663(2001); Deltcheva E. et al., Nature 471:602-607(2011); and Jinek et al., Science 337:816-821(2012), the entire contents of each of which are incorporated herein by reference). Additional suitable Cas9 or Cpf1 nucleases and sequences will be apparent to those of skill in the art based on this disclosure, and such Cas9 or Cpf1 nucleases and sequences include Cas9 sequences from the organisms and loci disclosed in Chylinski et al., (2013) RNA Biology 10:5, 726-737, incorporated herein by reference.

In some embodiments, the Cas9 used herein is from Streptococcus pyogenes (Uniprot Reference Sequence: Q99ZW2, SEQ ID NO:1)

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKERGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVETSGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITWIERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD (single underline: HNH domain;
double underline: RuvC domain).

In some embodiments, Cpf1 nuclease from Francisella novicida is used (FnCpf1, Uniport Reference Sequence: A0Q7Q2)

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKEKNLENQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKENTIIGGKEVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN

In some embodiments, the Cas9 nuclease used herein is from Streptococcus Aureus.

MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

In some embodiments, the Cas9 nuclease used herein is from Streptococcus thermophilus (Streptococcus thermophilus wild type CRISPR3 Cas9, St3Cas9)

MTKPYSIGLDIGTNSVGWAVITDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKVYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEV
FKDDTKNGYAGYIDGKTNQEDFYVYLKNLLAEFEGADYFLEKIDREDFLR
KQRTFDNGSIPYQIELQEMRAILDKQAKEYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNFMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSKIDNNALQNDRLYLYYLQNGKDMYTGDDLDIDRLSNYDIDHIIP
QAFLKDNSIDNKVLVSSASNRGKSDDFPSLEVVKKRKTFWYQLLKSKLIS
QRKFDNLTKAERGGLLPEDKAGFIQRQLVETRQITKHVARLLDEKFNNKK
DENNRAVRTVKIITLKSTLVSQFRKDFELYKVREINDFHHAHDAYLNAVI
ASALLKKYPKLEPEFVYGDYPKYNSFRERKSATEKVYFYSNIMNIFKKSI
SLADGRVIERPLIEVNEETGESVWNKESDLATVRRVLSYPQVNVVKKVEE
QNHGLDRGKPKGLFNANLSSKPKPNSNENLVGAKEYLDPKKYGGYAGISN
SFAVLVKGTIEKGAKKKITNVLEFQGISILDRINYRKDKLNFLLEKGYKD
IELIIELPKYSLFELSDGSRRMLASILSTNNKRGEIHKGNQIFLSQKFVK
LLYHAKRISNTINENHRKYVENHKKEFEELFYYILEFNENYVGAKKNGKL
LNSAFQSWQNHSIDELCSSFIGPTGSERKGLFELTSRGSAADFEFLGVKI
PRYRDYTPSSLLKDATLIHQSVTGLYETRIDLAKLGEG

In some embodiments, the Cas9 nuclease used herein is from Streptococcus thermophilus (Streptococcus thermophilus CRISPR1 Cas9 wild type, St1Cas9)

MSDLVLGLDIGIGSVGVGILNKVTGEIIHKNSRIFPAAQAENNLVRRTNR
QGRRLTRRKKHRRVRLNRLFEESGLITDFTKISINLNPYQLRVKGLTDEL
SNEELFIALKNMVKHRGISYLDDASDDGNSSIGDYAQIVKENSKQLETKT
PGQIQLERYQTYGQLRGDFTVEKDGKKHRLINVFPTSAYRSEALRILQTQ
QEFNPQITDEFINRYLEILTGKRKYYHGPGNEKSRTDYGRYRTSGETLDN
IFGILIGKCTFYPDEFRAAKASYTAQEFNLLNDLNNLTVPTETKKLSKEQ
KNQIINYVKNEKAMGPAKLFKYIAKLLSCDVADIKGYRIDKSGKAEIHTF
EAYRKMKTLETLDIEQMDRETLDKLAYVLTLNTEREGIQEALEHEFADGS
FSQKQVDELVQFRKANSSIFGKGWHNFSVKLMMELIPELYETSEEQMTIL
TRLGKQKTTSSSNKTKYIDEKLLTEEIYNPVVAKSVRQAIKIVNAAIKEY
GDFDNIVIEMARETNEDDEKKAIQKIQKANKDEKDAAMLKAANQYNGKAE
LPHSVFHGHKQLATKIRLWHQQGERCLYTGKTISIHDLINNSNQFEVDHI
LPLSITFDDSLANKVLVYATANQEKGQRTPYQALDSMDDAWSFRELKAFV
RESKTLSNKKKEYLLTEEDISKEDVRKKEIERNLVDTRYASRVVLNALQE
HFRAHKIDTKVSVVRGQFTSQLRRHWGIEKTRDTYHHHAVDALIIAASSQ
LNLWKKQKNTLVSYSEDQLLDIETGELISDDEYKESVFKAPYQHFVDTLK
SKEFEDSILFSYQVDSKFNRKISDATIYATRQAKVGKDKADETYVLGKIK
DIYTQDGYDAFMKIYKKDKSKFLMYRHDPQTFEKVIEPILENYPNKQINE
KGKEVPCNPFLKYKEEHGYIRKYSKKGNGPEIKSLKYYDSKLGNHIDITP
KDSNNKVVLQSVSPWRADVYFNKTTGKYEILGLKYADLQFEKGTGTYKIS
QEKYNDIKKKEGVDSDSEFKFTLYKNDLLLVKDTETKEQQLFRFLSRTMP
KQKHYVELKPYDKQKFEGGEALIKVLGNVANSGQCKKGLGKSNISIYKVR
TDVLGNQHIIKNEGDKPKLDF

In some embodiments, Cas9 refers to Cas9 from: Corynebacterium ulcerans (NCBI Refs: NC_015683.1, NC_017317.1); Corynebacterium diphtheria (NCBI Refs: NC 016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC 021846.1); Streptococcus iniae (NCBI Ref: NC_021314.1); Belliella baltica (NCBI Ref: NC_018010.1); Psychroflexus torquisI (NCBI Ref: NC_018721.1); Listeria innocua (NCBI Ref: NP 472073.1), Campylobacter jejuni (NCBI Ref: YP_002344900.1) or Neisseria. meningitidis (NCBI Ref: YP_002342100.1). Any known Cas9 or Cpf1 nucleases that cleaves a target DNA sequence in a programmable manner may be used in accordance with the present disclosure.

To use a Cas9 or Cpf1 nuclease for genome-editing, the Cas9 or Cpf1 nuclease needs to be in complex with a guide RNA (gRNA) that targets the nuclease to a target site in the genome. A “guide RNA,” as used herein, refers to a RNA molecule that can target (i.e., guide) a programmable nuclease (e.g., Cas9) to its target sequence. A gRNA comprises a Specificity Determining Sequence (SDS), which specifies the DNA sequence to be targeted, and is immediately followed by a 80 nucleotide (nt) scaffold sequence, which associates the gRNA with Cas9. In some embodiments, the SDS is about 20 nucleotides long. For example, the SDS may be 15, 16, 17, 18, 19, 20, 21, 22, 23, 24, or 25 nucleotides long. At least a portion of the target DNA sequence needs to be complementary to the SDS of the gRNA. In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence. In some embodiments, the gRNA comprises a structure 5′-[SDS]-[scaffold sequence]-3′. In some embodiments, the scaffold sequence comprises the nucleotide sequence of 5′-guuuuagagcuagaaauagcaaguuaaaauaaaggcuaguccguuaucaacuugaaaaaguggcaccgagucggugcuu uuu-3′. Other suitable scaffold sequences that may be used in accordance with the present disclosure are provided in Table 1.

TABLE 1
Guide RNA Scaffold Sequences

| Organism | gRNA handle sequence |
|---|---|
| S. pyogenes | GUUUAAGAGCUAUGCUGGAAAGCCACGGUGA |
| | AAAAGUUCAACUAUUGCCUGAUCGGAAUAAA |
| | UUUGAACGAUACGACAGUCGGUGCUUUUUUU |
| S. pyogenes | GUUUAAGAGCUAGAAAUAGCAAGUUUAAAUA |
| | AGGCUAGUCCGUUAUCAACUUGAAAAAGUGG |
| | CACCGAGUCGGUGCUUUUUU |
| S. | GUUUUUGUACUCUCAAGAUUCAAUAAUCUUG |
| thermophilus | CAGAAGCUACAAAGAUAAGGCUUCAUGCCGA |
| CRISPR1 | AAUCAACACCCUGUCAUUUUAUGGCAGGGUG |
| | UUUU |
| S. | GUUUUAGAGCUGUGUUGUUUGUUAAAACAAC |
| thermophilus | ACAGCGAGUUAAAAUAAGGCUUAGUCCGUAC |
| CRISPR3 | UCAACUUGAAAAGGUGGCACCGAUUCGGUGU |
| | UUUU |
| C. jejuni | AAGAAAUUUAAAAAGGGACUAAAAUAAAGAG |
| | UUUGCGGGACUCUGCGGGGUUACAAUCCCCU |
| | AAAACCGCUUUU |
| F. novicida | AUCUAAAAUUAUAAAUGUACCAAAUAAUUAA |
| | UGCUCUGUAAUCAUUUAAAAGUAUUUUGAAC |
| | GGACCUCUGUUUGACACGUCUGAAUAACUAA |
| | AA |
| S. | UGUAAGGGACGCCUUACACAGUUACUUAAAU |
| thermophilus | CUUGCAGAAGCUACAAAGAUAAGGCUUCAUG |
| 2 | CCGAAAUCAACACCCUGUCAUUUUAUGGCAG |
| | GGUGUUUUCGUUAUUU |
| M. mobile | UGUAUUUCGAAAUACAGAUGUACAGUUAAGA |
| | AUACAUAAGAAUGAUACAUCACUAAAAAAAG |
| | GCUUUAUGCCGUAACUACUACUUAUUUUCAA |
| | AAUAAGUAGUUUUUUUU |
| L. innocua | AUUGUUAGUAUUCAAAAUAACAUAGCAAGUU |
| | AAAAUAAGGCUUUGUCCGUUAUCAACUUUUA |
| | AUUAAGUAGCGCUGUUUCGGCGCUUUUUUU |
| S. pyogenes | GUUGGAACCAUUCAAAACAGCAUAGCAAGUU |
| | AAAAUAAGGCUAGUCCGUUAUCAACUUGAAA |
| | AAGUGGCACCGAGUCGGUGCUUUUUUU |
| S. mutans | GUUGGAAUCAUUCGAAACAACACAGCAAGUU |
| | AAAAUAAGGCAGUGAUUUUUAAUCCAGUCCG |
| | UACACAACUUGAAAAAGUGCGCACCGAUUCG |
| | GUGCUUUUUUAUUU |
| S. | UUGUGGUUUGAAACCAUUCGAAACAACACAG |
| thermophilus | CGAGUUAAAAUAAGGCUUAGUCCGUACUCAA |
| | CUUGAAAAGGUGGCACCGAUUCGGUGUUUUU |
| | UUU |
| N. | ACAUAUUGUCGCACUGCGAAAUGAGAACCGU |
| meningitidis | UGCUACAAUAAGGCCGUCUGAAAAGAUGUGC |
| | CGCAACGCUCUGCCCCUUAAAGCUUCUGCUU |
| | UAAGGGGCA |
| P. multocida | GCAUAUUGUUGCACUGCGAAAUGAGAGACGU |
| | UGCUACAAUAAGGCUUCUGAAAAGAAUGACC |
| | GUAACGCUCUGCCCCUUGUGAUUCUUAAUUG |
| | CAAGGGGCAUCGUUUUU |

In some embodiments, the guide RNA is about 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence.

For Cas9 to successfully bind to the DNA target sequence, a region of the target sequence must be complementary to the SDS of the gRNA sequence and must be immediately followed by the correct protospacer adjacent motif (PAM) sequence (e.g., NGG for Cas9 and TTN, TTTN, or YTN for Cpf1). The specific structure of the guide nucleotide sequences depends on its target sequence and the relative distance of a PAM sequence downstream of the target sequence.

A protospacer adjacent motif (PAM) is typically a sequence of nucleotides located adjacent to (e.g., within 10, 9, 8, 7, 6, 5, 4, 3, 3, or 1 nucleotide(s) of a target sequence). A PAM sequence is “immediately adjacent to” a target sequence if the PAM sequence is contiguous with the target sequence (that is, if there are no nucleotides located between the PAM sequence and the target sequence). In some embodiments, a PAM sequence is a wild-type PAM sequence. Examples of PAM sequences include, without limitation, NGG, NGR, NNGRR(T/N), NNNNGATT, NNAGAAW, NGGAG, and NAAAAC, AWG, CC. In some embodiments, a PAM sequence is obtained from Streptococcus pyogenes (e.g., NGG or NGR). In some embodiments, a PAM sequence is obtained from Staphylococcus aureus (e.g., NNGRR(T/N)). In some embodiments, a PAM sequence is obtained from Neisseria meningitidis (e.g., NNNNGATT). In some embodiments, a PAM sequence is obtained from Streptococcus thermophilus (e.g., NNAGAAW or NGGAG). In some embodiments, a PAM sequence is obtained from Treponema denticola NGGAG (e.g., NAAAAC). In some embodiments, a PAM sequence is obtained from Escherichia coli (e.g., AWG). In some embodiments, a PAM sequence is obtained from Pseudomonas auruginosa (e.g., CC). Other PAM sequences are contemplated. A PAM sequence is typically located downstream (i.e., 3′) from the target sequence, although in some embodiments a PAM sequence may be located upstream (i.e., 5′) from the target sequence.

In some embodiments, the genome-editing agent encapsulated in the nanoparticles of the present disclosure is a nucleic acid (e.g., an expression vector) encoding a Cas9 protein and/or a gRNA. The Cas9 protein and the gRNA may be encoded by a single nucleic acid or by two separate nucleic acids. In some embodiments, the genome-editing agent encapsulated in the nanoparticles of the present disclosure is an isolated Cas9/gRNA complex. Being “isolated” means a molecule (e.g., Cas9 or gRNA) that is isolated from, or is otherwise substantially free of (e.g., at least 80%, 90%, 95%, 97%, or 99% free of), other substances (e.g., other proteins or other nucleic acids). One skilled in the art is familiar with methods of protein and/or nucleic acid isolation or purification. A Cas9 and a gRNA may be isolated individually and combined to form a complex in vitro, or co-expressed in a cell to allow complex formation before isolation.

In some embodiments, delivery of a genome-editing agent (e.g., a CRISPR/Cas system described herein) to a cell (e.g., a cancer cell) results in the targeting of the genome-editing agent to a target gene (e.g., a Cas9 nuclease may be targeted by the gRNA to a target gene). A “target gene” refers to a gene within the genome of the cell (e.g., a cancer cell) targeted and cleaved by the genome-editing nuclease (e.g., Cas9 nuclease). In some embodiments, the target gene is in the genome of a mammal. In some embodiments, the target gene in the genome of a human. In some embodiments, the target gene in the genome of a non-human animal.

In some embodiments, once the Cas9 nuclease is targeted to the target gene by the gRNA, the Cas9 “edits” the target gene. “Edit” means the Cas9 nuclease introduces a double-strand DNA break in the target gene, which is repaired through nonhomologous end-joining (NHEJ) or homologous recombination (HR), resulting in insertion, deletion, or replacement of nucleotides in the target gene (i.e., edits).

In some embodiments, the target gene is an oncogene. Any oncogenes described herein may be targeted by the genome-editing agent. In some embodiments, the oncogene is lipocalin 2 (Lcn2). In some embodiments, editing of the oncogene by a genome-editing agent (e.g., the CRISPR/Cas system) inactivates the oncogene. “Inactive a gene” means reducing the expression level or activity of a protein or nucleic acid molecule produced from the gene by at least 40%. For example, a gene is considered to be inactivated when the expression level or activity of a protein or nucleic acid molecule produced from the gene is reduced by at least 40%, at least 45%, at least 50%, at least 55%, at least 60%, at least 65%, at least 70%, at least 75%, at least 80%, at least 85%, at least 90%, at least 95%, at least 99%, or 100%. In some embodiments, a gene is considered to be inactivated when the expression level or activity of a protein or nucleic acid molecule produced from the gene is reduced by 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, inactivation of an oncogene treats cancer.

The nanoparticles or delivery systems of the present disclosure may be formulated in pharmaceutical compositions. In some embodiments, the pharmaceutical composition further comprises a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the pharmaceutical composition can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The nanoparticle and/or the pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

Other aspects of the present disclosure provide methods of delivering an agent (e.g., a therapeutic agent or a genome-editing agent) to a cell, the methods comprising contacting the cell with the nanoparticle or the delivery system described herein. In some embodiments, the cell expresses a surface protein targeted by the ligand conjugated on the surface of the nanoparticle, leading to specific binding of the nanoparticle to the cell and delivering of the agent to the cell. In some embodiments, the nanoparticle or the delivery system does not deliver the agent to a cell that does not express a surface protein targeted by the ligand conjugated on the surface of the nanoparticle.

In some embodiments, the cell is a mammalian cell. In some embodiments, the cell is a human cell. In some embodiments, the cell is a cultured cell. In some embodiments, the cell is a cell in vivo in a subject. In some embodiments, the cell is a cancer cell. In some embodiments, the cancer cell overexpresses EGFR and/or ICAM-1 on its surface. In some embodiments, the cancer cell is a breast cancer cell. In some embodiments, the cancer cell is a triple-negative breast cancer (TNBC) cell.

Some aspects of the present disclosure relate to methods of editing a target gene in the genome of a subject. In some embodiments, the method comprises administer to the subject an effective amount of the nanoparticle of delivery system comprising a genome-editing agent. In some embodiments, the target gene may be associated with a disease or disorder. One skilled in the art is familiar with genes that are associated with diseases or disorders (e.g., genetic disorder or cancer). In some embodiments, editing of the gene that is associated with a disease or disorder results in an edited gene that that is not associated with the disease or disorder.

Further provided herein are methods of treating a disease or disorder, the method comprising administering a therapeutically effective amount of a nanoparticle or delivery system described herein to a subject in need thereof, wherein the nanoparticle or delivery system comprises a therapeutic agent encapsulated in the nanoparticle. One skilled in the art is able to identify the therapeutic agent to be used based on the disease or disorder that is being treated.

In some embodiments, the disease or disorder is cancer. Non-limiting, exemplary cancers include: neoplasms, malignant tumors, metastases, or any disease or disorder characterized by uncontrolled cell growth such that it would be considered cancerous. The cancer may be a primary or metastatic cancer. Cancers include, but are not limited to, adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, biliary tract cancer, osteosarcoma, fibrous histiocytoma, brain cancer, brain stem glioma, cerebellar astrocytoma, malignant glioma, glioblastoma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, acute lymphocytic and myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, choriocarcinoma, hematological neoplasm, adult T-cell leukemia, lymphoma, lymphocytic lymphoma, stromal tumors and germ cell tumors, or Wilms tumor. In some embodiments, the cancer is lung cancer, breast cancer, prostate cancer, colorectal cancer, gastric cancer, liver cancer, pancreatic cancer, brain and central nervous system cancer, skin cancer, ovarian cancer, leukemia, endometrial cancer, bone, cartilage and soft tissue sarcoma, lymphoma, neuroblastoma, nephroblastoma, retinoblastoma, or gonadal germ cell tumor.

In some embodiments, the cancer is selected from the group consisting of: breast cancer, pancreatic cancer, brain and central nervous system cancer, skin cancer, ovarian cancer, leukemia, endometrial cancers, bone, cartilage and soft tissue sarcomas, lymphoma, neuroblastoma, nephroblastoma, retinoblastoma, and gonadal germ cell tumors. In some embodiments, the cancer is triple negative breast cancer.

In some embodiments, the methods described herein delivers therapeutic agents specifically to a cancer cell. In some embodiments, the methods described herein are effective in reducing tumor size, slowing rate of tumor growth, reducing cell proliferation of the tumor, promoting cancer cell death, inhibiting angiogenesis, inhibiting metastasis, or otherwise improving overall clinical condition, without necessarily eradicating the cancer. In some embodiments, the compositions and methods described herein are effective in eradicating the cancer.

In some embodiments, the compositions and methods of the present disclosure, when administered to the subject, prevents metastasis of the cancer. The term “metastasis” refers to the spread of a primary tumor from one organ or part of the body to another not directly connected with it. A “primary tumor” refers to a tumor growing at the anatomical site where tumor progression began and proceeded to yield a cancerous mass. Most cancers develop at their primary site but then go on to spread to other parts of the body, i.e., metastasis. These further tumors are secondary tumors. Metastasis results from several interconnected processes including cell proliferation, angiogenesis, cell adhesion, migration, and invasion into the surrounding tissue. The term “prevent metastasis” means the process of a primary to spread to other parts of the body that is not directly connected is inhibited, or that the development of the secondary tumor is prevented.

The term “inhibits growth and/or proliferation” (e.g., referring to cancer or tumor cells) is intended to include any measurable decrease in the growth of a cell when contacted with a cancer-targeting liposome as compared to the growth of the same cell not in contact with the cancer-targeting liposome, e.g., the inhibition of growth of a cell by at least about 10%, 20%, 30%, 40%, 50%, 60%, 70%, 80%, 90%, 99%, or 100%).

The term “reduce tumor size,” as used herein, refers to the decrease in tumor size compared to before the subject was treated using the methods and the compositions of the present disclosure. In some embodiments, the tumor size is reduced by at least 10%, at least 20%, at least 30%, at least 50%, at least 60%, at least 70%, at least 80%, at least 90%, at least 99%. In some embodiments, the tumor size is reduced by 100%, i.e., the tumor disappears. In some embodiments, the tumor is reduced to no more that 80%, no more than 70%, no more than 60%, no more than 40%, no more than 30%, no more than 20%, no more than 10% no more than 5%, no more than 1%, or no more than 0.1% of its original size. The term “kills cancer cells” means causing death to cancer cells, e.g., via apoptosis or necrosis.

In its broadest sense, the terms “treatment” or “to treat” refer to both therapeutic and prophylactic treatments. If the subject in need of treatment has cancer, then “treating the condition” refers to ameliorating, reducing or eliminating one or more symptoms associated with the cancer or the severity of cancer or preventing any further progression of cancer. If the subject in need of treatment is one who is at risk of having cancer, then treating the subject refers to reducing the risk of the subject having cancer or preventing the subject from developing cancer.

A subject shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof. A subject in need thereof can be a subject who has a risk of developing cancer (i.e., via a genetic test) or a subject who has cancer.

Pharmaceutically compositions that may be used in accordance with the present disclosure may be directly administered to the subject or may be administered to a subject in need thereof in a therapeutically effective amount. The term “therapeutically effective amount” refers to the amount necessary or sufficient to realize a desired biologic effect. For example, a therapeutically effective amount of a cancer-target liposome associated with the present disclosure may be that amount sufficient to ameliorate one or more symptoms of cancer. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular pharmaceutically compositions being administered the size of the subject, or the severity of the disease or condition. One of ordinary skill in the art can empirically determine the effective amount of a particular therapeutic compound associated with the present disclosure without necessitating undue experimentation.

Subject doses of the cancer-targeting liposomes or liposome drug delivery systems described herein for delivery typically range from about 0.1 μg to 10 mg per administration, which depending on the application could be given daily, weekly, or monthly and any other amount of time there between. In some embodiments a single dose is administered during the critical consolidation or reconsolidation period. The doses for these purposes may range from about 10 μg to 5 mg per administration, and most typically from about 100 μg to 1 mg, with 2-4 administrations being spaced, for example, days or weeks apart, or more. In some embodiments, however, parenteral doses for these purposes may be used in a range of 5 to 10,000 times higher than the typical doses described above.

In some embodiments, a cancer-targeting liposome or liposome drug delivery system of the present disclosure is administered at a dosage of between about 1 and 10 mg/kg of body weight of the mammal. In other embodiments a cancer-targeting liposome or liposome drug delivery system of the present disclosure is administered at a dosage of between about 0.001 and 1 mg/kg of body weight of the mammal. In yet other embodiments, a cancer-targeting liposome or liposome drug delivery system of the present disclosure is administered at a dosage of between about 10-100 ng/kg, 100-500 ng/kg, 500 ng/kg-1 mg/kg, or 1-5 mg/kg of body weight of the mammal, or any individual dosage therein.

The formulations of the present disclosure are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compound associated with the present disclosure can be administered to a subject by any mode that delivers the therapeutic agent or compound to the desired surface, e.g., mucosal, injection to cancer, systemic, etc. Administering the pharmaceutical composition of the present disclosure may be accomplished by any means known to the skilled artisan. Preferred routes of administration include but are not limited to oral, parenteral, intravenous, intramuscular, intranasal, sublingual, intratracheal, inhalation, ocular, vaginal, rectal and intracerebroventricular.

For oral administration, the pharmaceutically compositions of the present disclosure can be formulated readily by combining the active compound(s) with pharmaceutically acceptable carriers well known in the art. Such carriers enable the compounds of the present disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The pharmaceutical compositions can be included in the formulation as fine multi particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The pharmaceutical compositions of the present disclosure, when desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The pharmaceutical compositions of the present disclosure and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the present disclosure contain an effective amount of a therapeutic compound of the present disclosure optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present disclosure, and with each other, in a manner such that there is no interaction which would substantially impair the desired pharmaceutical efficiency.

The pharmaceutical compositions may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells are sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipophilic compounds to the brain. For instance, DHA, a fatty acid naturally occurring in the human brain has been found to be useful for delivering drugs covalently attached thereto to the brain (Such as those described in U.S. Pat. No. 6,407,137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7,119,074 describes amphiphilic prodrugs of a therapeutic compound conjugated to an PEG-oligomer/polymer for delivering the compound across the blood brain barrier. Others are known to those of skill in the art.

The pharmaceutical compositions of the present disclosure may be delivered with other therapeutics for treating cancer.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The present disclosure is further illustrated by the following Examples, which in no way should be construed as further limiting. The entire contents of all of the references (including literature references, issued patents, published patent applications, and co pending patent applications) cited throughout this application are hereby expressly incorporated by reference.

### Examples

### Design and Synthesis of TNLG

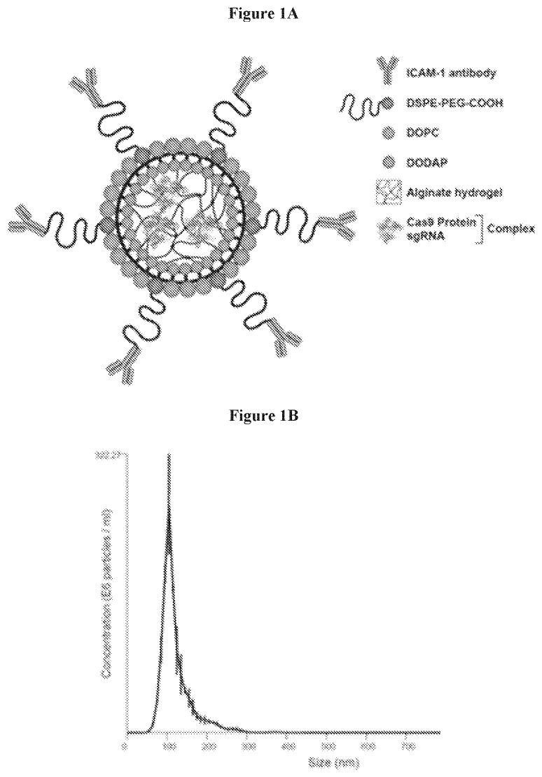

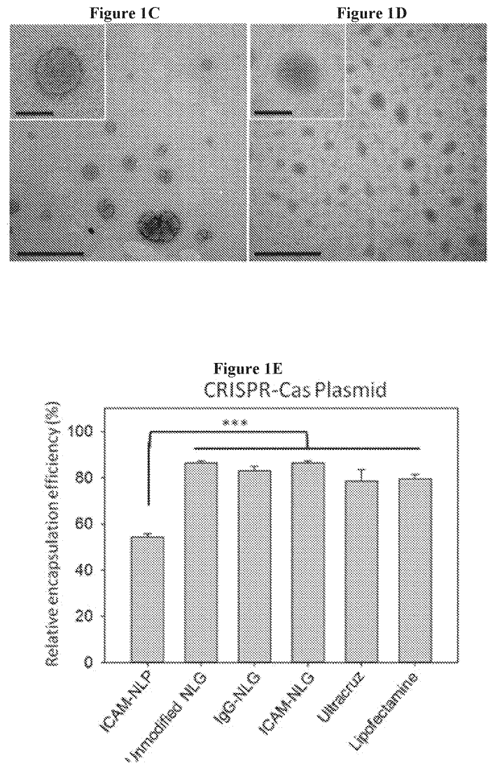

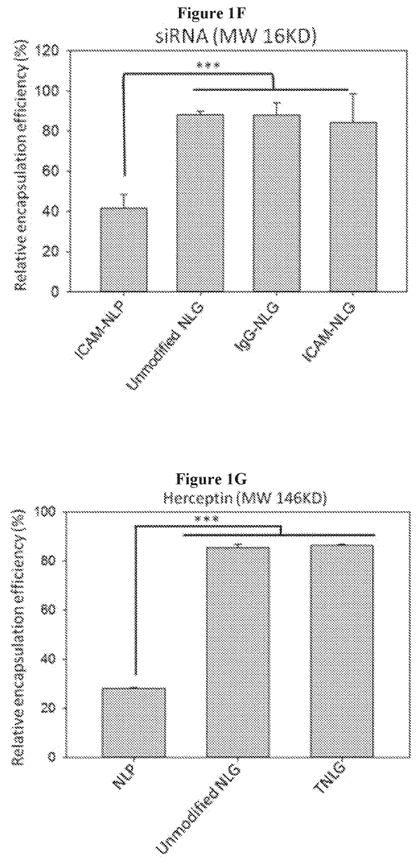

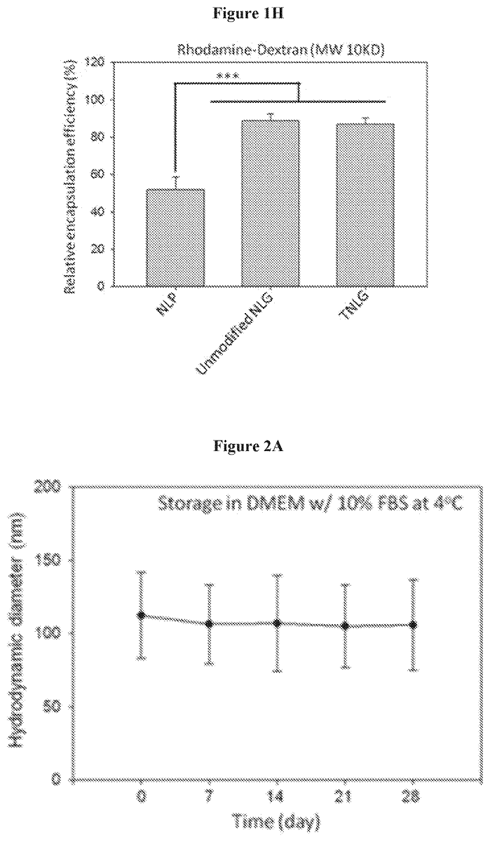

Recently, several groups have demonstrated the use of nanoscale drug delivery system (nanoDDS) for CRIPSR-Cas9 genome engineering. Their nanoDDSs are involved in using cationic polymers (PEI derivatives) or lipids (DOTAP), which are widespread used in siRNA/DNA delivery. An unneglectable fact is that the toxicity of cationic polymer/lipid may hinder clinical applications of CRIPSR-Cas9 mediated gene therapy. In contrast, the invention of the present disclosure selected non-viral, non-cationic TNLGs (structure shown in FIG. 1A) to enhance CRIPSR-Cas9 plasmid delivery to TNBC cells. TNLGs are composed of TNBC-targeting ligand (ICAM-1 antibody)-conjugated, unilamellar 100 nm liposomes (outer shell) and CRISPR-Cas9 plasmid-encapsulating alginate hydrogel (inner core). It was thought that this unique liposome-hydrogel complex structure of TNLG can provide a polymer network that efficiently confine and retain macromolecules such as CRISPR-Cas9 plasmid (MW˜120 KD) and, in turn, improve its encapsulation efficiency and release profile. TNLGs were prepared by the extrusion method. Briefly, lipids (85 mol % dioleoylphophatidylcholine (DOPC, liquid phase), 5 mol % dioleoyldimethylammonium propane (DODAP, a pH sensitive lipid), 10 mol % 1,2-distearoyl-sn-glycero-3-phosphoethanolamine-N-[carboxy(polyethylene glycol) (DSPE-PEG(2 k)-COOH, liquid phase)) were dissolved at their respective ratios in chloroform and dried in a rotary evaporator under reduced pressure. The lipid film was hydrated in 1 mg/mL sodium alginate solution, vortexed, exposed to 10 cycles of freeze/thaw, and subjected to a series of nanoporous membrane extrusions in the order of 400, 200, and 100 nm polycarbonate track-etched membranes. Series extrusion is a critical step in engineering nanoscale (size<200 nm) TNLGs because it is extremely difficult to directly extrude unextruded lipid/hydrogel solution through a nanoporous membrane with a pore size of 100 or 200 nm. The series extrusion step overcomes this technical difficulty and significantly improves the efficiency of generating uniform and monodisperse TNLGs with a size less than 200 nm. Encapsulation of CRISPR-Cas9 plasmid is achieved by addition to the sodium alginate solution prior to extrusion. Extrusion is followed by dialysis (300 k MWCO) to remove external CRISPR-Cas9 plasmid. After dialysis, the resulting nanolipogels are crosslinked with 2 mg/mL CaCl₂ solution and covalently conjugated to either IgG or ICAM1 antibodies. Unconjugated antibodies are removed using dialysis (1,000 k MWCO). The density of IgG or ICAM1 antibody is quantified by flow cytometry with reference to Quantum Simply Cellular microbeads, which have defined numbers of antibody binding sites per bead. The successful synthesis of TNLGs was confirmed by TEM (FIGS. 1C and 1D), and its size and zeta potential were determined by dynamic light scattering (FIG. 1B, ZetaPals, Brookhaven). The encapsulation efficiency was determined using a standard fluorimetric assay (Picogreen, Invitrogen). The TNLG systems of the present disclosure demonstrated a significant higher CRISPR-Cas9 plasmid encapsulation efficiency (FIG. 1E, over 80%) than traditional liposomes (approximately 40-60%). It was also confirmed that TNLGs also had similar high encapsulation efficiency for siRNAs (FIG. 1F), proteins (e.g. Herceptin, FIG. 1G), and polymers (e.g. Rhodamine-dextran, FIG. 1H).

### Stability and Cytotoxicity of TNLGs

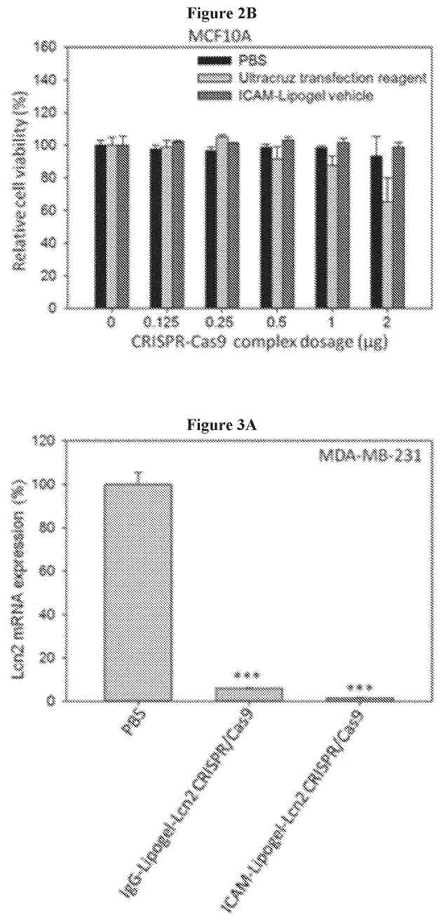

The serum stability of nanolipogel was investigated by incubating it within 10% fetal bovine serum (FBS) supplemented cell cultured medium (DMEM). The dynamic light scattering measurements showed the hydrodynamic diameter of nanolipogel remained unchanged during one month incubation (FIG. 2A), suggesting nanolipogel is a stable delivery system for intravenous administration. The cytotoxicity of nanolipogel was evaluated in normal human breast MCF10A cells (FIG. 2B), and showed no cytotoxicity at optimized gene-editing dosage ranges (0-2 mg/mL of CRISPR-Cas9 plasmid).

CRIPSR/Cas9 Gene Editing Efficiency of TNBC Cells Treated with TNLGs

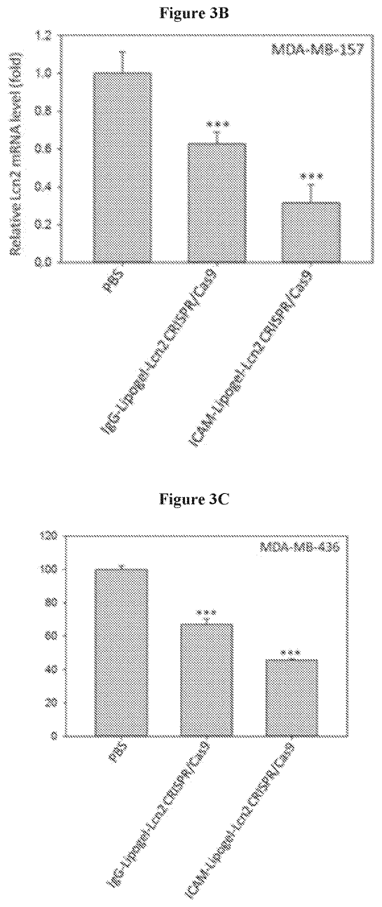

The gene-editing efficiency of engineered nanolipogels loaded with CRISPR-Cas9 plasmid using qRT-PCR. In pilot studies, lipocalin 2 (Lcn2), a well-established oncogene, was used as the therapeutic target, and sgRNA/Cas9 nanolipogel was used to knockout Lcn2 from three human TNBC cell lines (MDA-MB-231, MDA-MB-436, and MDA-MB-157). In FIG. 3, the engineered TNBC-targeted sgRNA/Cas9 nanolipogels demonstrated a significant Lcn2 knockout efficiency of 60-98% in three TNBC cells.

### Determine the Therapeutic Benefits of Lcn2 Gene Knockout in TNBC Cell Using TNLGs

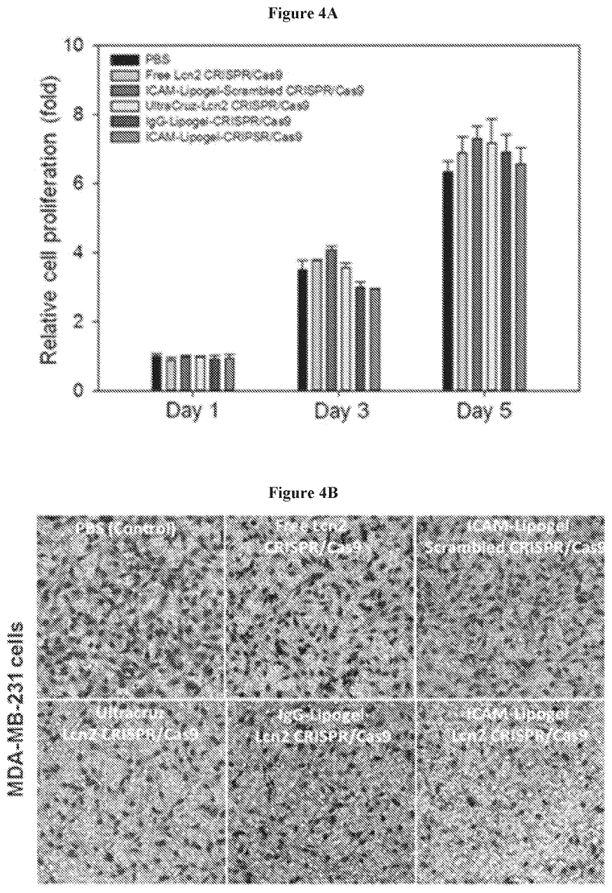

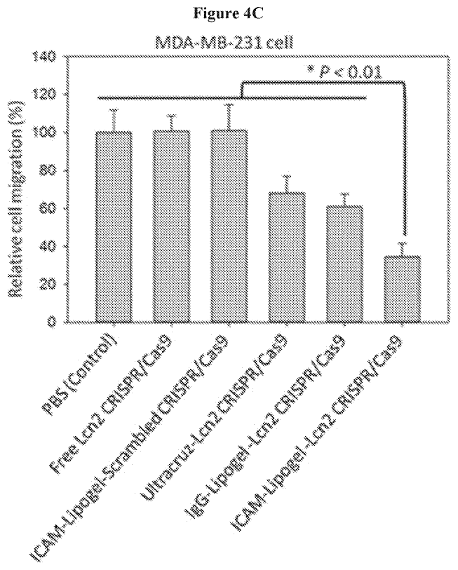

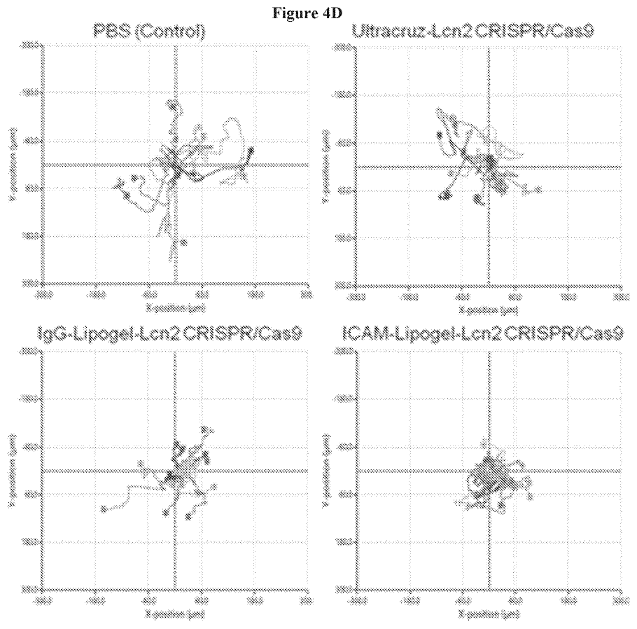

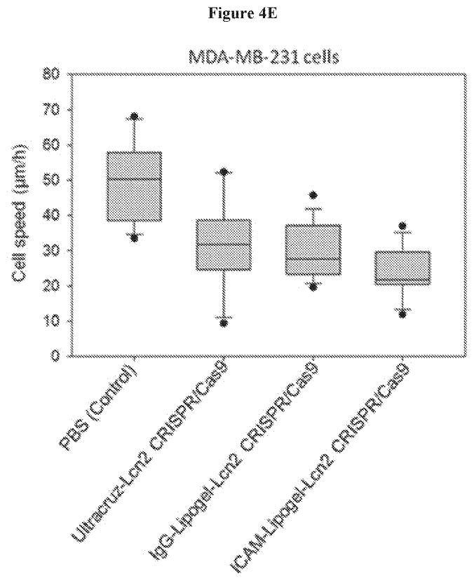

The therapeutic effects of this gene editing were evaluated by assessing triple negative breast cancer (TNBC) cells' two predominant malignant behaviors: proliferation and migration. As shown from cell proliferation studies, Lcn2 knockout by CRISPR-Cas9 gene editing system via TNLGs did not alter MDA-MB-231 cell proliferation (FIG. 4A). However, Lcn2 knockout did exhibit potent activity in inhibiting MDA-MB-231 cell migration via blocking Lcn2 signaling cascades (FIGS. 4B and C). The number of migrated MDA-MB-231-Lcn2 KO cells was significantly reduced by 60%, in comparison to untreated cells. These cell migration results correlated with MDA-MB-231 cell mobility changes after Lcn2 KO (FIGS. 4D and 4E). Lcn2 KO by TNLGs significantly impeded MDA-MB-231 cell mobility by over 60%. These findings demonstrate that tumor-targeted gene editing on TNBC cells may be useful in blocking metastasis of TNBC.

### Evaluate Orthotopic Tumor Accumulation of TNLG

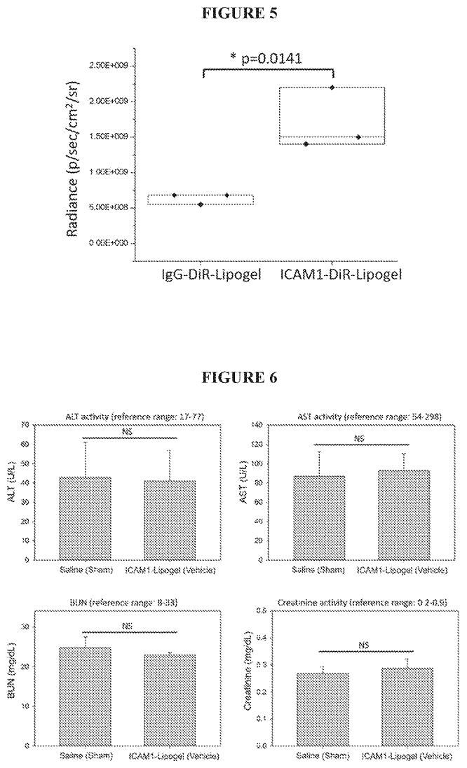

The in vivo tumor-targeting activity of TNLGs was evaluated using near infrared (NIR) fluorescent imaging in an orthotopic TNBC tumor model (FIG. 5). ICAM1 antibody-conjugated lipogels were labeled with DiR, a NIR lipid dye, (ICAM1-DiR-Lipogel) and were intravenously injected into MDA-MB-231 tumor bearing mice at a dosage of 20 mg lipids/kg mouse weight. IgG-DiR-Lipogel was used as a non-targeting control. In vivo NIR imaging was performed at 48 h post-injection. Quantified NIR signals confirmed that the tumor accumulation of ICAM1-DiR-LP was significantly higher (˜2.7-fold) than that of IgG-DiR-Lipogel at 48 h after a single tail vein administration. These results indicate that the engineered TNLG can selectively deliver significantly more CRISPR-Cas gene editing systems to TNBC tumors in vivo than conventional non-targeting drug delivery systems.

### Evaluate Systematic Toxicity of TNLG

The systematic cytotoxicity of TNLG treatment was evaluated via blood chemistry analysis. ICAM1 antibody conjugated lipogels (ICAM1-Lipogel, vehicle) were intravenously injected into healthy nude mice at a dosage of 20 mg lipids/kg mouse weight. PBS was used as a control. At the time point of 48 h post-injection, the serum from each group was collected and aspartate aminotransferase (AST), alanine aminotransferase (ALT), creatinine, and blood urea nitrogen (BUN) were measured to evaluate their systematic toxicity. As shown in FIG. 6, it was found that the ICAM1-Lipogel did not induce any elevation in the levels of all tested biomarkers. These in vivo data demonstrate that TNLG at 20 mg lipids/kg dosage exhibited no systemic toxicity.

The foregoing written specification is considered to be sufficient to enable one skilled in the art to practice the disclosure. The present disclosure is not to be limited in scope by examples provided, since the examples are intended as a single illustration of one or more aspects of the disclosure and other functionally equivalent embodiments are within the scope of the disclosure.

Various modifications of the disclosure in addition to those shown and described herein will become apparent to those skilled in the art from the foregoing description and fall within the scope of the appended claims. The advantages and objects of the disclosure are not necessarily encompassed by each embodiment of the disclosure.

### ACKNOWLEDGEMENT

The support from the Breast Cancer Research Foundation in making this invention is acknowledged.

What is claimed is:

1. A nanoparticle comprising:
(i) a non-cationic liposome;
(ii) a ligand conjugated to the liposome surface; and
(iii) a hydrogel encapsulated in the liposome.

2. The nanoparticle of claim 1, wherein the non-cationic liposome comprises a neutral lipid.

3. The nanoparticle of claim 1 or 2, wherein the non-cationic liposome does not comprise a cationic lipid.

4. The nanoparticle of claim 3, wherein the neutral lipid is 1,2-dioleoyl-sn-glycero-3-phosphocholine (DOPC).

5. The nanoparticle of any one of claims 1-4, wherein the nanoparticle comprises an anionic lipid.

6. The nanoparticle of any one of claims 1-5, wherein the liposome further comprises a pH-responsive lipid.

7. The nanoparticle of claim 6, wherein the pH-responsive lipid comprises 1,2-dioleoyl-3-dimethylammoniumpropane (DODAP).

8. The nanoparticle of any one of claims 1-7, wherein the liposome further comprises a functionalized lipid.

9. The nanoparticle of claim 8, wherein the functionalized lipid is a lipid-polymer conjugate.

10. The nanoparticle of claim 6, wherein the lipid-polymer conjugate is a lipid-polyethylene glycol (PEG) conjugate.

11. The nanoparticle of any one of claims 8-10, wherein the functionalized lipid comprises a carboxylic acid at the distal end of the lipid.

12. The nanoparticle of claim 11, wherein the functionalized lipid is 1,2-distearoyl-sn-glycero-3-phosphoethanolamine-N-[carboxy(polyethyleneglycol)-2000]-COOH (DSPE-PEG-COOH).

13. The nanoparticle of any one of claims 8-12, wherein the functionalized lipid is up to 10% of total lipids in the liposome.

14. The nanoparticle of claim 1, wherein the liposome comprises DOPC, DODAP, and DSPE-PEG-COOH.

15. The nanoparticle of claim 14, wherein the ratio of DOPC:DODAP:DSPE-PEG-COOH is 85:5:10.

16. The nanoparticle of claim 15, wherein the hydrogel comprises sodium alginate.

17. The nanoparticle of any one of claims 1-16, wherein the nanoparticle has a diameter of no more than 200 nm.

18. The nanoparticle of any one of claims 1-17, wherein the ligand targets a cell surface protein.

19. The nanoparticle of any one of claims 1-18, wherein the ligand is selected from the group consisting of: antibodies, antibody fragments, synthetic peptides, natural ligands, and aptamers.

20. The nanoparticle of claim 19, wherein the ligand is an antibody.

21. The nanoparticle of claim 20, wherein the antibody is an ICAM-1 antibody.

22. The nanoparticle of any one of claims 1-21, wherein the nanoparticle further comprises a second ligand conjugated to the liposome surface.

23. The nanoparticle of claim 22, wherein the second ligand targets a second cell surface protein.

24. The nanoparticle of claim 22 or 23, wherein the second ligand is selected from the group consisting of: antibodies, antibodies fragments, synthetic peptides, natural ligands, aptamers.

25. The nanoparticle of claim 24, wherein the second ligand is an antibody.

26. The nanoparticle of claim 25, wherein the second antibody is an EGFR antibody.

27. The nanoparticle of any one of claims 1-26, further comprising an agent encapsulated in the liposome.

28. The nanoparticle of claim 27, wherein the agent is a therapeutic agent.

29. The nanoparticle of claim 28, wherein the therapeutic agent is an anti-cancer agent.

30. The nanoparticle of claim 28 or claim 29, wherein the therapeutic agent is selected from the group consisting of: small molecules, oligonucleotides, polypeptides, and combinations thereof.

31. The nanoparticle of claim 27, wherein the agent comprises a genome-editing agent.

32. The nanoparticle of claim 31, wherein the agent comprises a nucleic acid encoding a Cas9 protein and a guide RNA (gRNA).

33. The nanoparticle of claim 31, wherein the agent comprises an isolated Cas9/gRNA complex.

34. The nanoparticle of claim 32 or claim 33, wherein the gRNA targets the Cas9 protein to a target gene.

35. The nanoparticle of claim 34, wherein the Cas9 edits the target gene.

36. The nanoparticle of claim 34, wherein the target gene is an oncogene.

37. The nanoparticle of claim 35, wherein the oncogene is lipocalin 2 (Lcn2).

38. The nanoparticle of claim 35 or claim 36, wherein editing of the oncogene by Cas9 inactivates the oncogene.

39. A composition comprising the nanoparticle of any one of claims 1-38.

40. A delivery system, the delivery system comprising:
(i) a non-cationic liposome;
(ii) a ligand conjugated to the liposome surface;
(iii) a hydrogel encapsulated in the liposome; and
(iv) a genome-editing agent encapsulated in the liposome.

41. The delivery system of claim 40, wherein the non-cationic liposome comprises a neutral lipid.

42. The delivery system of claim 40 or claim 41, wherein the non-cationic liposome does not comprise a cationic lipid.

43. The delivery system of claim 42, wherein the neutral lipid is 1,2-dioleoyl-sn-glycero-3-phosphocholine (DOPC).

44. The delivery system of any one of claims 40-43, wherein the nanoparticle comprises an anionic lipid.

45. The delivery system of any one of claims 40-44, wherein the liposome further comprises a pH-responsive lipid.

46. The delivery system of claim 45, wherein the pH-responsive lipid comprises 1,2-dioleoyl-3-dimethylammoniumpropane (DODAP).

47. The delivery system of any one of claims 40-46, wherein the liposome further comprises a functionalized lipid.

48. The delivery system of claim 47, wherein the functionalized lipid is a lipid-polymer conjugate.

49. The delivery system of claim 45, wherein the lipid-polylmer conjugate is a lipid-polyethylene glycol (PEG) conjugate.

50. The delivery system of any one of claims 47-49, wherein the functionalized lipid comprises a carboxylic acid at the distal end of the lipid.

51. The delivery system of claim 50, wherein the functionalized lipid is 1,2-distearoyl-sn-glycero-3-phosphoethanolamine-N-[carboxy(polyethylene glycol)-2000]-COOH (DSPE-PEG-COOH).

52. The delivery system of any one of claims 47-51 herein the functionalized lipid is up to 10% of total lipids in the liposome.

53. The delivery system of claim 40, wherein the liposome comprises DOPC, DODAP, and DSPE-PEG-COOH.

54. The delivery system of claim 53, wherein the ratio of DOPC:DODAP:DSPE-PEG-COOH is 85:5:10.

55. The delivery system of any one of claims 40-54, wherein the hydrogel comprises sodium alginate.

56. The delivery system of any one of claims 40-55, wherein the nanoparticie has a diameter of less than 200 nm.

57. The delivery system of any one of claims 40-56, wherein the ligand targets a cell surface protein.

58. The delivery system of any one of claims 40-57, wherein the ligand is selected from the group consisting of: antibodies, antibodies fragments, synthetic peptides, natural ligands, aptamers.

59. The delivery system of claim 58, wherein the ligand is an antibody.

60. The delivery system of claim 59, wherein the antibody is an ICAM-1 antibody.

61. The delivery system of any one of claims 40-60, wherein the nanoparticie further comprises a second ligand conjugated to the liposome surface.

62. The delivery system of claim 61, wherein the second ligand targets a second cell surface protein.

63. The delivery system of claim 61 or 62, wherein the second ligand is selected from the group consisting of: antibodies, antibodies fragments, synthetic peptides, natural ligands, aptamers.

64. The delivery system of claim 63, wherein the second ligand is an antibody.

65. The delivery system of claim 64, wherein the second antibody is an EGFR antibody.

66. The delivery system of any one of claims 40-65, wherein the genome-editing agent comprises a nucleic acid encoding a Cas9 protein and a guide RNA (gRNA).

67. The delivery system of any one of claims 40-65, wherein the genome-editing agent comprises an isolated Cas9/gRNA complex.

68. The delivery system of claim 66 or claim 67, wherein the gRNA targets the Cas9 protein to a target gene.

69. The delivery system of claim 68, wherein the Cas9 edits the target gene.

70. A composition comprising the delivery system of any one of claims 40-69.

71. A method of delivering an agent to a cell, the method comprising contacting the cell with the nanoparticle of any one of claims 27-38, or the delivery system of any one of claims 40-69, wherein the cell expresses a surface protein targeted by the ligand on the nanoparticle, and wherein the contacting results in delivery of the agent to the cell.

72. The method of claim 71, wherein the cell is a mammalian cell.

73. The method of claim 72 wherein the cell is a human cell.

74. The method of claim 72 or claim 73, wherein the cell is a cultured cell.

75. The method of claim 72 or claim 73, wherein the cell is a cell in vivo in a subject.

76. The method of any one of claims 71-75, wherein the cell is a cancer cell.

77. The method of claim 76, wherein the cancer cell is a triple negative breast cancer cell (TNBC).

78. A method of treating a disease or disorder, the method comprising administering a therapeutically effective amount of a delivery system to a subject in need thereof, wherein the delivery system comprises the nanoparticle of any one of claims 1-26 and a therapeutic agent encapsulated in the nanoparticle.

79. The method of claim 78, wherein the disease or disorder is cancer.

80. The method of claim 79, wherein the cancer is selected from the group consisting of: breast cancer, pancreatic cancer, brain and central nervous system cancer, skin cancer, ovarian cancer, leukemia, endometrial cancers, bone, cartilage and soft tissue sarcomas, lymphoma, neuroblastoma, nephroblastoma, retinoblastoma, and gonadal germ cell tumors.

81. The method of claim 79, wherein the cancer is triple negative breast cancer (TNBC).

82. The method of any one of claims 78-81, wherein the delivery system is administered orally, parenterally, intramuscularly, intranasally, intratracheal, intracerebroventricularly, intravenously, or intraperitoneally.

83. A method of editing a target gene in the genome of a subject, the method comprising administering to the subject an effective amount of the delivery system of any one of claims 40-69.

84. The method of claim 83, wherein the target gene is associated with a disease or disorder, and wherein editing the target gene results in an edited gene that is not associated with the disease or disorder.


SEQUENCE LISTING (20 sequences)

SEQ ID NO: 1 - protein, 1368 aa, Streptococcus pyogenes [sequence omitted]

SEQ ID NO: 2 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 1053 aa, Streptococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1388 aa, Streptococcus thermophilus
MTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1121 aa, Streptococcus thermophilus
MSDLVLGLDIGIGSVGVGILNKVTGEIIHKNSRIFPAAQAENNLVRRTNRQGRRLTRRKKHRRVRLNRLFEESGLITDFTKISINLNPYQLRVKGLTDELSNEELFIALKNMVKHRGISYLDDASDDGNSSIGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 6 - RNA, 82 nt, Artificial Sequence
  Synthetic polynucleotide
guuuuagagcuagaaauagcaaguuaaaauaaaggcuaguccguuaucaacuugaaaaaguggcaccgagucggugcuuuuu

SEQ ID NO: 7 - RNA, 93 nt, Streptococcus pyogenes
guuuaagagcuaugcuggaaagccacggugaaaaaguucaacuauugccugaucggaauaaauuugaacgauacgacagucggugcuuuuuuu

SEQ ID NO: 8 - RNA, 82 nt, Streptococcus pyogenes
guuuaagagcuagaaauagcaaguuuaaauaaggcuaguccguuaucaacuugaaaaaguggcaccgagucggugcuuuuuu

SEQ ID NO: 9 - RNA, 97 nt, Streptococcus thermophilus
guuuuuguacucucaagauucaauaaucuugcagaagcuacaaagauaaggcuucaugccgaaaucaacacccugucauuuuauggcaggguguuuu

SEQ ID NO: 10 - RNA, 97 nt, Streptococcus thermophilus
guuuuagagcuguguuguuuguuaaaacaacacagcgaguuaaaauaaggcuuaguccguacucaacuugaaaagguggcaccgauucgguguuuuu

SEQ ID NO: 11 - RNA, 74 nt, Campylobacter jejuni
aagaaauuuaaaaagggacuaaaauaaagaguuugcgggacucugcgggguuacaauccccuaaaaccgcuuuu

SEQ ID NO: 12 - RNA, 95 nt, Francisella novicida
aucuaaaauuauaaauguaccaaauaauuaaugcucuguaaucauuuaaaaguauuuugaacggaccucuguuugacacgucugaauaacuaaaa

SEQ ID NO: 13 - RNA, 109 nt, Streptococcus thermophilus
uguaagggacgccuuacacaguuacuuaaaucuugcagaagcuacaaagauaaggcuucaugccgaaaucaacacccugucauuuuauggcaggguguuuucguuauuu

SEQ ID NO: 14 - RNA, 110 nt, Mycoplasma mobile
uguauuucgaaauacagauguacaguuaagaauacauaagaaugauacaucacuaaaaaaaggcuuuaugccguaacuacuacuuauuuucaaaauaaguaguuuuuuuu

SEQ ID NO: 15 - RNA, 92 nt, Listeria innocua
auuguuaguauucaaaauaacauagcaaguuaaaauaaggcuuuguccguuaucaacuuuuaauuaaguagcgcuguuucggcgcuuuuuuu

SEQ ID NO: 16 - RNA, 89 nt, Streptococcus pyogenes
guuggaaccauucaaaacagcauagcaaguuaaaauaaggcuaguccguuaucaacuugaaaaaguggcaccgagucggugcuuuuuuu

SEQ ID NO: 17 - RNA, 107 nt, Streptococcus mutan
guuggaaucauucgaaacaacacagcaaguuaaaauaaggcagugauuuuuaauccaguccguacacaacuugaaaaagugcgcaccgauucggugcuuuuuuauuu

SEQ ID NO: 18 - RNA, 96 nt, Streptococcus thermophilus
uugugguuugaaaccauucgaaacaacacagcgaguuaaaauaaggcuuaguccguacucaacuugaaaagguggcaccgauucgguguuuuuuuu

SEQ ID NO: 19 - RNA, 102 nt, Neisseria meningitidi
acauauugucgcacugcgaaaugagaaccguugcuacaauaaggccgucugaaaagaugugccgcaacgcucugccccuuaaagcuucugcuuuaaggggca

SEQ ID NO: 20 - RNA, 110 nt, Pasteurella multocid
gcauauuguugcacugcgaaaugagagacguugcuacaauaaggcuucugaaaagaaugaccguaacgcucugccccuugugauucuuaauugcaaggggcaucguuuuu